(19) United States
(12) Patent Application Publication
(10) Pub. No.: US 2023/0330207 A1
(43) Pub. Date: Oct. 19, 2023

(54) CANCER TREATMENT UTILIZING PRE-EXISTING MICROBIAL IMMUNITY

(71) Applicant: THE USA, as represented by the Secretary, Department of Health and Human Services, Bethesda, MD (US)

(72) Inventors: John T. SCHILLER, Kensington, MD (US); Nicolas CUBURU, Bethesda, MD (US); Douglas R. LOWY, Bethesda, MD (US)

(21) Appl. No.: 18/346,680

(22) Filed: Jul. 3, 2023

Related U.S. Application Data
(62) Division of application No. 16/760,138, filed on Apr. 29, 2020.
(60) Provisional application No. 62/582,097, filed on Nov. 6, 2017.

(51) Int. Cl. A61K 39/12; A61P 35/00; C12N 15/86

(52) U.S. Cl. CPC A61K 39/12; A61P 35/00; C12N 15/86; A61K 2039/505

(57) ABSTRACT

Methods, compositions, and kits for redirecting a preexisting immune response in an individual to reduce or stabilize a cancer in the individual.

### CROSS REFERENCE TO RELATED APPLICATIONS

This application is a national stage application under 35 U.S.C. 371 and claims the benefit of PCT Application No. PCT/US2018/059384 having an international filing date of 6 Nov. 2018, which designated the United States, which PCT application claimed the benefit of U.S. Provisional Patent Application Ser. No. 62/582,097, filed Nov. 6, 2017, the disclosures of each of which are incorporated herein by reference.

### REFERENCE TO SEQUENCE LISTING

This application contains a Sequence Listing submitted as an electronic text file named “060734_696158_Corrected_SequenceListing_ST25.txt,” having a size of 17 KB, and created on Feb. 18, 2022. The information contained in this electronic file is hereby incorporated by reference in its entirety pursuant to 37 CFR § 1.52(e)(5). No new matter has been added.

### TECHNICAL FIELD

The present invention relates to immunology and cancer therapy, including methods, compositions, and kits for directing a patient's existing immune response to a cancer.

### BACKGROUND

Persistent asymptomatic viral infections are usually controlled by cell-mediated and/or humoral immunity in healthy individuals but can be reactivated in immune compromised individuals. Cell-mediated immunity against some chronic viral infection increases with age and leads to induction of many fully functional virus-specific T cells. Cytomegalovirus (CMV) is a β-herpesvirus that is highly prevalent globally (infecting 50-90% of human populations) and mostly asymptomatic in healthy individuals. CMV establishes a life-long persistent infection that requires long-lived cellular immunity to prevent disease. Consequently, CMV reactivation is a threat in the context of immune suppression, e.g. in hematopoietic stem cell transplant. In immunocompetent individuals, CD4 and CD8 T cell responses against CMV display broad reactivity and high magnitude against multiple CMV antigens, with high prevalence in the general human population, and increase with age (M. Bajwa et al., J Infect Dis 215, 1212-20 (2017)). Memory inflation is a hallmark of persistent CMV infection and has been extensively studied in humans. CMV-specific CD8+ T cell responses can be divided in two types depending on whether they expand with time (inflationary) or remain stationary upon resolution of primary infection (non-inflationary) (G. A. O'Hara, Trends Immunol 33:84-90 (2012)). The nature of the antigen and the pattern of antigen expression during persistent CMV infection leads to CD8+ T cells that harbor a memory phenotype (non-inflationary) or effector phenotype (inflationary). Mouse CMV infection also establishes life-long persistent infection with induction of immune responses that mimic those to CMV in humans (Id).

Induction of anti-tumor T cell responses is paramount in the development of effective immunotherapies against cancer. Only a subset of cancer patients responds to current immunotherapy. Generating T cell immunity against cancer antigens often requires highly personalized approaches or relying on preexisting anti-cancer T cells. It is also difficult to generate potent de novo T cell immunity in cancer patients, particularly in the elderly. Personalized approaches rely on vaccines against tumor associated antigens, neoantigens (i.e. mutated self-antigens), or viral oncoproteins. Other approaches are based on adoptive transfer of chimeric antigen receptor transduced T cells or infusion of monoclonal antibodies which require the laborious identification of tumor-specific antigens and are applicable to only a subset of cancer types or subtypes. Finally, adoptive transfer of tumor specific lymphocytes expanded ex vivo is a methodology that aims to take advantage of naturally-occurring anti-tumor responses. All these approaches are highly personalized and require the identification tumor epitopes and/or expansion of patient autologous cells ex vivo.

In parallel, in situ tumor immunotherapy based on cytokines or TLR ligands have been used but mostly target innate immune recognition mechanisms to change the tumor immune microenvironment, to trigger immunogenic cancer cell death and to favor epitope spreading.

Therefore, a simple, broadly applicable, antigen agnostic, immunotherapy methodology is still needed to harness the effects of the immune system in early and long-term cancer control through direct killing and promotion of epitope spreading, respectively.

### SUMMARY

The present inventors have recognized that the complex adaptive cell-mediated immunity that develops over many years to strongly control a chronic viral infection in an aging person is the type of cellular-mediated immunity that is effective at controlling tumor growth. To harness this type of antiviral immunity to treat cancer, the inventors have developed a new approach to in situ immunotherapy by targeting directly the tumor environment with highly functional preexisting antiviral T cells using tumor-tropic papillomavirus pseudovirions or by in situ injection of minimal viral CD8 and CD4 T-cell cytomegalovirus (CMV) epitopes. Presentation of viral epitopes in the tumor environment results in the recruitment and activation of viral antigen-specific T cells in situ, resulting in the killing of otherwise viral-negative tumor cells and changes in the tumor microenvironment. This approach responds to an unmet need as it fulfils all criteria for successful immunotherapy by promoting and establishing both early and long-term cancer cell killing and epitope spreading.

Thus, this disclosure provides methods of treating cancer in an individual by recruiting a preexisting immune response to the site of the cancer, thereby treating the cancer.

The preexisting immune response may be an immune memory response that exists in the individual prior to diagnosis with cancer. The preexisting, immune response may be a naturally-occurring, preexisting immune response.

In these methods, recruiting the preexisting immune response to a cancer cell may include introducing into the cancer an antigen that is not expressed by the cancer cell prior to the initiation of treatment, wherein the antigen is recognized by one or more components of the preexisting immune response.

These methods may include confirming that the individual has a preexisting immune response to the antigen, prior to introducing the antigen into the tumor. These methods may also include evaluating the individual's preexisting immune response to the antigen. In these methods, confirming the presence of the preexisting immune response may include identifying a T-cell response to the antigen in a sample from the individual.

In these methods, introducing the antigen may include injecting the antigen into the cancer. Additionally or alternatively, introducing the antigen may be accomplished by introducing into the cancer a nucleic acid molecule encoding the antigen. In these methods, the nucleic acid molecule may be DNA or RNA. For the use of RNA, the RNA may be modified so that it is more resistant to degradation. The nucleic acid molecule may be introduced into the cancer cells by injection. Additionally or alternatively, the nucleic acid molecule may be introduced into the cancer using a viral vector or a pseudovirion such as a papillomavirus pseudovirion.

In these methods, the antigen may be a viral antigen. For example, the antigen may be a polypeptide comprising at least one epitope from a cytomegalovirus (CMV) protein, which is recognized by the one or more components of the preexisting immune response. In these methods, the CMV protein may be selected from the group consisting of pp50, pp65, pp150, IE-1, IE-2, gB, US2, US6, UL16, and UL18. The polypeptide may be a 9-15 mer MHC I-restricted peptide. Alternatively or additionally, the polypeptide may be an at least a 15-mer MHC II-restricted peptide. Alternatively or additionally, the antigen comprises a sequence at least 90% identical to a sequence selected from the sequences of SEQ ID NOS: 1-67. In these methods, the one or more components of the immune response may be T-cells.

In these methods, recruitment of the preexisting immune response may alter the microenvironment of the cancer.

In these methods, the antigen may be administered in combination with an agent that augments the immune response. Exemplary agents include an agent selected from a TLR agonist; an IL-1R8 cytokine antagonist; intravenous immunoglobulin (IVIG); peptidoglycan isolated from gram positive bacteria; lipoteichoic acid isolated from gram positive bacteria; lipoprotein isolated from gram positive bacteria; lipoarabinomannan isolated from mycobacteria, zymosan isolated from yeast cell wall; polyadenylic-polyuridylic acid; poly (IC); lipopolysaccharide; monophosphoryl lipid A; flagellin; Gardiquimod; Imiquimod; R848; oligonucleosides containing CpG motifs, a CD40 agonist, and 23S ribosomal RNA. In exemplary methods, the antigen may be administered in combination with poly-IC.

Another aspect provides kits for testing a patient and recruiting a preexisting immune response to the site of a cancer in the patient. These kits may include at least one CMV peptide antigen or a nucleic acid encoding the peptide, a pharmaceutically acceptable carrier, a container, and a package insert or label indicating the administration of the CMV peptide, for reducing at least one symptom of the cancer in the patient.

This Summary is neither intended nor should it be construed as being representative of the full extent and scope of the present invention. Moreover, references made herein to “the present disclosure,” or aspects thereof, should be understood to mean certain embodiments of the present invention and should not necessarily be construed as limiting all embodiments to a particular description. The present disclosure is set forth in various levels of detail in this Summary as well as in the attached drawings and the Description of Embodiments and no limitation as to the scope of the present disclosure is intended by either the inclusion or non-inclusion of elements, components, etc. in this Summary. Additional aspects of the present invention will become readily apparent from the Detailed Description, particularly when taken together with the figures.

### BRIEF DESCRIPTION OF FIGURES

FIG. 1A shows that murine cytomegalovirus (mCMV) infection induces a massive cytokine response against a mCMV peptide pool. FIG. 1B shows IFN-gamma production by spleen CD4+ and CD8+ T cells after peptide re-stimulation with indicated MHC-I and MHC-II restricted mCMV peptides.

FIG. 2A shows an injection protocol for intratumoral transduction of solid tumors with HPV Psv expressing mCMV antigens. FIGS. 2B and 2C show tumor volume following intratumoral injection of HPV16 Psv expressing m122 and m45, or HPV Psv expressing red fluorescent protein (RFP), respectively.

FIG. 3A depicts the injection protocol for intratumoral transduction of solid tumors with HPV Psv expressing mCMV antigens in combination with poly(I:C) (PIC). FIGS. 3B-3E show that this intratumoral transduction protocol slows tumor growth. FIGS. 3F and 3G show the infiltration of tumors by E7-, m45- and m122-specific CD8+ T cells, analyzed by MHC-I tetramer staining and FACS.

FIG. 4A shows the effects on survival, and FIG. 4B shows the effect on tumor growth following intratumoral injection of MCMV MHC-I restricted peptides in C57Bl/6 mice infected with murine cytomegalovirus (mCMV).

FIG. 5 shows the effects of different doses of intratumoral injection of mCMV MHC-I restricted peptides on tumor growth in C57Bl/6 mice infected with murine cytomegalovirus (mCMV).

FIGS. 6A and 6B show the effects of intratumoral injection of combinations of mCMV MHC-I and MHC-II restricted peptides on tumor growth in C57Bl/6 mice infected with mCMV. FIG. 6C shows E7-, m45-, m122-specific CD8+ T cell responses in blood as analyzed by FACS using MHC-I tetramers for each peptide, demonstrating that sequential intratumoral inoculation with mCMV CD4 and then CD8 epitopes preferentially induces anti-tumor immunity.

FIG. 7 shows the effect of complete clearance of primary tumors on long term protection against secondary tumor challenge.

FIG. 8 shows that mCMV infection induces an inflationary CD8+ T cell response in C57BL/6 mice.

FIG. 9A shows inflationary and non-inflationary CD8+ T cells produce IFN-γ and CD4+ T cells produce IFN-γ. FIG. 9B shows cytokine production by mCMV CD8+ T cells to MHC-I restricted peptide pool.

FIG. 10A shows the experimental protocol timing for the mouse TC1 tumor model for the intratumoral administration of mCMV peptides. FIGS. 10B and 10C show the distribution of mCMV-specific CD8+ T cells in tumor-bearing mice. Inflationary (IE3; FIG. 10B) and non-inflationary (m45; FIG. 10C) specific CD8+ T cells were detected by FACS using MHC-I tetramer staining.

FIG. 11A shows the experimental protocol timing for the mouse TC1 tumor model used for gene expression analysis of tumor microenvironment. FIGS. 11B-11F show tumor infiltration by CD45+ cells (FIG. 11B), Th1 cells (FIG. 11C), cytotoxic CD8 T cells (FIG. 11D), NK cells (FIG. 11E), or dendritic cells (FIG. 11F) after intratumoral treatment.

FIGS. 12A and 12B show intratumoral injection of mCMV CD8 epitopes delays tumor growth Poly(I:C) co-injection improves tumor control. FIG. 12A shows the effects of intratumoral injection of MHC-I restricted mCMV peptide alone +/−poly(I:C). FIG. 12B shows the effects of an intratumoral injection of MHC-I restricted mCMV peptide titration.

FIGS. 13A and 13B show protection from TC1 tumor challenge by intratumoral injection of mCMV MHC-I and/or MHC-II peptides with poly(I:C). Sequential intratumoral inoculation with CD4 then CD8 MCMV epitopes suppresses tumor growth (FIG. 13A) and promotes long-term survival (FIG. 13B).

FIG. 14 shows E7 tetramer positive CD8+ T Cell responses in blood after 6 treatments with MHC-I restricted selected m38, m45, and m122 peptide, and/or MHC-II restricted m139 selected peptide with or without poly(I:C)(30 ug), and saline or poly(I:C) alone as controls.

FIG. 15 shows that complete clearance of primary tumors confers long term protection against secondary tumor challenge.

FIG. 16 shows protection from MC38 tumor challenge by intratumoral injection of mCMV MHC-I and MHC-II peptides with poly(I:C).

### DETAILED DESCRIPTION

The present invention relates to a novel method of treating cancer. Specifically, the present invention relates to a method of treating cancer in an individual, utilizing the individual's own immune system to attack cancer cells. The method makes use of the fact that individuals possess preexisting immune responses that were not originally elicited in response to a cancer, but that were elicited instead by microorganisms in the environment. Because cancer cells would not normally express the microbial antigens that elicited the preexisting immune response, it would not be expected that such an immune response would attack a cancer. However, the inventors have discovered that such preexisting immune responses can be recruited to attack a cancer. One way this can be achieved is by introducing into the cancer, one or more antigens recognized by the preexisting immune response, resulting in cells of the immune response attacking antigen-displaying cancer cells. Thus, these methods are not directed to cancer cells that express the antigen prior to the treatment of the cancer patient. For example, many glioblastoma cancer cells are found to express CMV antigens, and the methods of this disclosure would not be used to treat such glioblastomas using the individual's preexisting immunity to CMV. Further, destruction of cancer cells can result in components of the preexisting immune response being exposed to cancer cell antigens. This can result in elicitation of an immune response against the cancer cell antigens. Thus, a general method of the invention can be practiced by recruiting a preexisting immune response in an individual to the site of a cancer, such that the preexisting immune response attacks the cancer. Recruitment may be achieved for example, by introducing into the cancer at least one antigen that is recognized by components (e.g., T-cells) of the individual's preexisting immune response.

The invention is not limited to particular embodiments described herein, as such may vary. The terminology used herein is for the purpose of describing particular embodiments only, and is not intended to be limiting.

As used herein, and in the appended claims, the singular forms “a,” “an,” and “the” include plural referents unless the context clearly dictates otherwise. For example, a nucleic acid molecule refers to one or more nucleic acid molecules. As such, the terms “a”, “an”, “one or more” and “at least one” can be used interchangeably. Similarly, the terms “comprising”, “including” and “having” can be used interchangeably. It is further noted that the claims may be drafted to exclude any optional element. As such, this statement is intended to serve as antecedent basis for use of such exclusive terminology as “solely,” “only” and the like regarding the recitation of claim elements, or use of a “negative” limitation.

Certain features of the invention, which are described in the context of separate embodiments, may also be provided in combination in a single embodiment. Conversely, various features of the invention, which are, for brevity, described in the context of a single embodiment, may also be provided separately or in any suitable sub-combination. All combinations of the embodiments are specifically embraced by the present invention and are disclosed herein just as if each and every combination was individually and explicitly disclosed. In addition, all sub-combinations are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates, which may need to be independently confirmed. All publications mentioned herein are incorporated herein by reference to disclose and describe the methods and/or materials in connection with which the publications are cited.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

One aspect is a method of treating cancer in an individual, comprising recruiting a preexisting immune response to a cancer, thereby treating the cancer.

As used herein, cancer refers to diseases in which abnormal cells divide without the appropriate control of cell division and/or cellular senescence. The term cancer is meant to encompass solid tumors as well as blood borne cancer. Generally, a tumor is an abnormal mass of tissue that usually does not contain a cyst or liquid area. Solid tumors may be benign (not life threatening), or malignant (life threatening). Different types of solid tumors are named for the type of cells that form them. Examples of solid tumors include sarcomas, carcinomas, and lymphomas. Blood cancers (also called hematologic cancers) are cancers that begin in blood-forming tissue, such as the bone marrow, or in the cells of the immune system. Examples of blood cancer include leukemia, lymphoma, and multiple myeloma.

In some cancers, the cells can invade tissues other than those from which the original cancer cells arose. In some cancers, cancer cells may spread to other parts of the body through the blood and lymph systems. Thus, cancers are usually named for the organ or type of cell in which they start. For example, a cancer that originates in the colon is called colon cancer; cancer that originates in melanocytes of the skin is called melanoma, etc. As used herein, cancer may refer to carcinomas, sarcomas, adenocarcinomas, lymphomas, leukemias, etc., including solid and lymphoid cancers, gastric, kidney cancer, breast cancer, lung cancer (including non-small cell and small cell lung cancer), bladder cancer, colon cancer, ovarian cancer, prostate cancer, pancreatic cancer, stomach cancer, brain cancer, head and neck cancers, skin cancer, uterine cancer, testicular cancer, esophageal cancer, liver cancer (including hepatocarcinoma), lymphoma, including non-Hodgkin's lymphomas (e.g., Burkitt's, Small Cell, and Large Cell lymphomas) and Hodgkin's lymphoma, leukemia, and multiple myeloma. In exemplary embodiments, the cancer is lung cancer or adenocarcinoma.

As used herein, the terms individual, subject, patient, and the like, are meant to encompass any mammal capable of developing cancer, with a preferred mammal being a human. The terms individual, subject, and patient by themselves do not denote a particular age, sex, race, and the like. Thus, individuals of any age, whether male or female, are intended to be covered by the present disclosure. Likewise, the methods of the present invention can be applied to any race of human, including, for example, Caucasian (white), African-American (black), Native American, Native Hawaiian, Hispanic, Latino, Asian, and European. Such characteristics may be significant. In such cases, the significant characteristic(s) (e.g., age, sex, race, etc.) will be indicated. These terms also encompass both human and non-human animals. Suitable non-human animals to test or treat for cancer include, but are not limited to companion animals (i.e. pets), food animals, work animals, or zoo animals.

As used herein, an immune, or immunological, response refers to the presence in an individual of a humoral and/or a cellular response to one or more antigens. For purposes of this disclosure, a “humoral response” refers to an immune response mediated by B-cells and antibody molecules, including secretory (IgA) or IgG molecules, while a “cellular response” is one mediated by T-lymphocytes and/or other white blood cells. One important aspect of cellular immunity involves an antigen-specific response by cytolytic T-cells (CTLs). CTLs have specificity for peptide antigens that are presented in association with proteins encoded by the major histocompatibility complex (MHC) on the surfaces of cells. CTLs help induce and promote the destruction of intracellular microbes, or the lysis of cells infected with such microbes. Another aspect of cellular immunity involves an antigen-specific response by helper T-cells. Helper T-cells act to help stimulate the function, and focus the activity, of nonspecific effector cells against cells displaying peptide antigens in association with MHC molecules on their surface. A cellular immune response also refers to the production of cytokines, chemokines and other such molecules produced by activated T-cells and/or other white blood cells, including those derived from CD4+ and CD8+ T-cells.

Thus, an immunological response may be one that stimulates CTLs, and/or the production or activation of helper T-cells. The production of chemokines and/or cytokines may also be stimulated. The immune response may also comprise an antibody-mediated immune response. Hence, an immunological response may include one or more of the following effects: the production of antibodies (e.g., IgA or IgG) by B-cells; and/or the activation of suppressor, cytotoxic, or helper T-cells, and/or T-cells directed specifically to an antigen. Such responses can be determined using standard immunoassays and neutralization assay, known in the art.

As used herein, a preexisting immune response is an immune response that is present in an individual prior to initiation of the cancer treatment. Thus, an individual having a preexisting immune response has an immune response against an antigen, prior to the initiation of a treatment using the antigen to treat cancer. A preexisting immune response can be a naturally occurring immune response, or it can be an induced immune response. As used herein, a naturally occurring preexisting immune response is an immune response in an individual that was elicited in response to an antigen, such as a bacterial or viral antigen, which the individual came into contact with unintentionally. That is, an individual having a preexisting immune response was not exposed to an antigen with the intent to generate an immune response to the antigen. An induced preexisting immune response is an immune response resulting from intentional exposure to an antigen, such as when receiving a vaccine. The preexisting immune response may be a naturally-occurring immune response, or the preexisting immune response may be an induced immune response.

As used herein, the phrase “recruiting an immune response,” refers to a process in which an antigen is administered to an individual such that components of a preexisting immune response travel through the body to the location where the antigen was administered, resulting in attack by the immune system components on cells displaying the antigen. As used herein, “components of an immune response” refers to cells that can bind to the antigen and initiate an immune response to the antigen. Antigens useful for practicing the invention are any molecules that can be recognized by cells of the preexisting immune system, particularly T-cells. One example of such a compound is a protein, such as a bacterial or viral protein.

As used herein, the phrase “treating a cancer” refers to various outcomes regarding a cancer. Treating a cancer includes reducing the rate of increase in the number of cancer cells in a treated individual. Such a reduction in the rate of increase can be due to a slowing in replication of cancer cells. Alternatively, the replication rate of cancer cells may be unaffected, an increase in the number of cancer cells may be killed by the preexisting immune response. In certain aspects, treating a cancer refers to a situation in which the number of cancer cells stops increasing, but remains at a constant level. Such a situation may arise due to inhibition of cancer cell replication by recruitment of the preexisting immune response, or it may be due to the rate of production of new cancer cells being balanced by the rate of cancer cell killing by the recruited preexisting immune response. Treating a cancer refers to stabilizing the cancer such that the growth of the cancer is decreased or stopped, or a decrease in the number of cancer cells in the treated individual, and/or in the individual being cancer free (i.e., no detectable cancer cells).

In embodiments, the step of recruiting the preexisting immune response comprises introducing into the cancer an antigen recognized by one or more components of the preexisting immune response. In preferred embodiments, the antigen is not present in the cancer prior to treatment. Thus, one embodiment is a method of treating a cancer in an individual, comprising recruiting a preexisting immune response to a cancer by introducing to the cancer an antigen recognized by one or more components of the preexisting immune response, wherein the antigen is not present in the cancer prior to treatment of the cancer. Thus, as noted above, the preexisting immune response may be a naturally-occurring immune response, or an induced immune response. Introduction of the antigen to the cancer can be achieved using methods known in the art, and can vary depending on the type of cancer being treated. For example, one type of cancer is a solid tumor. In such a cancer, the cancer cells replicate and remain adjacent to their parent cancer cell, resulting in the formation of a mass of tissue formed from the adjacent cancer cells. Because such cancers are masses of cells, the antigen can be delivered directly to, or into, the mass. One embodiment is a method of treating a cancer in an individual, wherein the cancer is a solid tumor, comprising recruiting a preexisting immune response to the solid tumor by introducing to the solid tumor an antigen recognized by one or more components of the preexisting immune response, wherein the antigen is not present in the solid tumor prior to treatment. In one embodiment, the preexisting immune response is a naturally-occurring immune response. In one embodiment, the preexisting immune response is an induced immune response. In one embodiment, the antigen is delivered to the cancer (e.g., solid tumor) by injection of the antigen into the cancer (e.g., solid tumor). In such an embodiment, the antigen is delivered directly into the cancer, allowing for the antigen to be displayed on MHC I molecules of the cells, either by direct binding to such molecules or by uptake and processing of the antigen by the cancer cells. In these methods, the antigen can be combined with other molecules or compounds that enhance uptake and/or presentation of the antigen to the immune system.

As previously described, in these methods the antigen may be a protein. These protein antigens may be injected directly into the cancer (e.g., tumor), as described above. Thus, one embodiment is a method of treating a cancer in an individual, wherein the cancer is a solid tumor, comprising recruiting a preexisting immune response to the solid tumor by injecting the solid tumor with an antigenic protein, wherein the antigenic protein is recognized by one or more components of the preexisting immune response, and wherein the antigenic protein is not present in the solid tumor prior to treatment. Alternatively, the protein antigen can be introduced to the cancer by introducing into the cancer a nucleic acid molecule encoding the protein. Thus, one embodiment is a method of treating a cancer in an individual, wherein the cancer is a solid tumor, comprising recruiting a preexisting immune response to the solid tumor by introducing to the solid tumor a nucleic acid molecule encoding an antigenic protein, wherein the antigenic protein is recognized by one or more components of the preexisting immune response, and wherein the antigenic protein is not present in the solid tumor prior to treatment. Introduction of the antigen-encoding nucleic acid molecule to the cancer can be performed using any suitable method known in the art. One embodiment is a method of treating a cancer in an individual, wherein the cancer is a solid tumor, comprising recruiting a preexisting immune response to the solid tumor by injecting a nucleic acid molecule encoding an antigenic protein into the solid tumor, wherein the antigenic protein is recognized by one or more components of the preexisting immune response, and wherein the antigenic protein is not present in the solid tumor prior to treatment. In these methods, the antigen-encoding nucleic acid molecule may be injected as a naked nucleic acid molecule (i.e., a nucleic acid molecule that is not complexed with other molecules intended to enhance delivery of stability of the nucleic acid molecule) or the injected antigen-encoding nucleic acid molecule may be complexed with one or more compounds intended to enhance delivery, stability, or longevity of the nucleic acid molecule. Examples of such compounds include lipids, proteins, carbohydrates, and polymers, including synthetic polymers.

Nucleic acid molecules encoding one more antigens can also be introduced to the cancer using a delivery vehicle, such as a recombinant virus or a pseudovirus (pseudovirion). Examples of viruses useful for practicing methods of the invention include, but are not limited to, adenoviruses, adeno-associated viruses, herpesviruses, and papillomaviruses. The use of such viruses to deliver nucleic acid molecules is known to those skilled in the art, and is also disclosed in U.S. Pat. No. 8,394,411, which is incorporated herein by reference. Examples of pseudoviruses useful for practicing methods of the invention include, but are not limited to, a hepatitis pseudovirus, an influenza pseudovirus, and a papilloma pseudovirus. As used herein, a pseudovirus refers to a particle comprising a virus capsid protein assembled into a virus-like particle (VLP) that is capable of binding to and entering a cancer cell. Such pseudovirion particles can, but preferably do not, package a sub-genomic amount of viral nucleic acid molecules. Methods of producing and using pseudovirions are known in the art, and are also described in U.S. Pat. Nos. 6,599,739; 7,205,126; and 6,416,945, all of which are incorporated herein by reference, in their entireties. Thus, this disclosure provides a method of treating a cancer in an individual, wherein the cancer is a solid tumor, comprising recruiting a preexisting immune response to the solid tumor by introducing to the tumor a recombinant virus, or pseudovirus, comprising a nucleic acid molecule encoding an antigenic protein, wherein the antigenic protein is recognized by one or more components of the preexisting immune response, and wherein the antigenic protein is not present in the solid tumor prior to treatment. Entry of a pseudovirus carrying a nucleic acid molecule of this disclosure into a cell results in expression of the encoded antigenic protein by the cell, and subsequent display of the antigen to the immune system. In these methods, the pseudovirus is a papilloma pseudovirus.

Introduction of viruses or pseudoviruses comprising an antigen-encoding nucleic acid molecule to a cancer can be achieved using any suitable method known in the art. For example, a recombinant virus, or pseudovirus, comprising the antigen-encoding nucleic acid molecule, can be injected near, or directly into, the cancer. Alternatively, a recombinant virus, or pseudovirus, comprising the antigen-encoding nucleic acid molecule, can be administered to the individual by a route that results in delivery of the recombinant virus, or pseudovirus, to the cancer. Examples of such routes include, but are not limited to, intravenous (IV) injection, intramuscular (IM) injection, intra-peritoneal (IP) injection, subcutaneous (SC) injection, and oral delivery. Thus, one embodiment is a method of treating a cancer in an individual, comprising administering to the individual a recombinant virus, or pseudovirus, comprising a nucleic acid molecule encoding an antigenic protein, wherein the cancer is a solid tumor, wherein the antigenic protein is recognized by one or more components of a preexisting immune response, and wherein the antigenic protein is not present in the solid tumor prior to treatment. In these methods, the recombinant virus, or pseudovirus, may be injected directly into the solid tumor, or the recombinant virus, or pseudovirus, may be delivered using a method selected from IV injection, IM injection, IP injection, SC injection, and oral delivery.

The methods of this disclosure can be used to treat blood borne cancers. Blood borne cancers, blood cancers, hematologic cancers, and the like, begin in blood-forming tissue, such as the bone marrow, or in the cells of the immune system. Examples of blood cancer include leukemia, lymphoma, and multiple myeloma. Such cancers begin when cells of blood forming tissue, or cells of the immune system, lose control of cellular replication and begin to replicate in an uncontrolled manner. Once formed, the blood cancer cells can make their way into the blood or lymphatic system, causing a significant rise in the number of cancer cells in the blood and/or the lymphatic system. For example, leukemia is a cancer found in the blood and bone marrow. Leukemia arises due to uncontrolled replication of white blood cells, resulting in a large increase in the number of abnormal white blood cells in the blood and lymph tissue. These abnormal white blood cells do not function properly and thus, individuals with leukemia are not able to fight infections. Thus, this disclosure provides a method of treating a hematologic cancer in an individual, comprising recruiting a preexisting immune response to hematologic cancer cells in the individual, by introducing to the hematologic cancer cells an antigen recognized by one or more components of a preexisting immune response, wherein the antigen is not present in, or on, the hematologic cancer cells prior to treatment. In these methods, the preexisting immune response may be a naturally-occurring immune response, or an induced immune response. Introduction of the antigen to the hematologic cancer cells can be performed using any suitable method. In these methods, the antigen may be introduced into the hematologic cancer cells by administering the antigen to the individual in a form that results in delivery of the antigen to the hematologic cancer cells. For example, the antigen can be administered to the individual using a method selected from IV injection, IM injection, IP injection, SC injection, and oral administration. In these methods, the antigen can be targeted to the hematologic cancer cell, for example by joining the antigen to a protein that binds a molecule on a hematologic cancer cell.

The antigen can also be introduced to the hematologic cancer cells by introducing a nucleic acid molecule encoding the antigenic protein to the hematologic cancer cells in the individual. Thus, this disclosure provides a method of treating a hematologic cancer in an individual, comprising recruiting a preexisting immune response to the hematologic cancer cells, by administering to the individual a nucleic acid molecule encoding an antigenic protein, wherein the antigenic protein is recognized by one or more components of a preexisting immune response, and wherein the antigenic protein is not present in, or on, the hematologic cancer cells prior to treatment. Administration of the antigen-encoding nucleic acid molecule to the individual can be performed using any suitable method known in the art. For example, the antigen-encoding nucleic acid molecule can be injected as a naked nucleic acid molecule. Alternatively or additionally, the antigen-encoding nucleic acid molecule may be complexed with one or more compounds intended to enhance delivery, stability, or longevity of the nucleic acid molecule. Examples of such compounds include lipids, proteins, carbohydrates, and polymers, including synthetic polymers.

Nucleic acid molecules encoding one more antigens can also be introduced to the hematologic cancer cells using a delivery vehicle, such as a recombinant virus or a pseudovirus. Examples of such delivery vehicles have been previously described herein. Examples of viruses useful for practicing methods of the invention include, but are not limited to, adenoviruses, adeno-associated viruses, herpesviruses, and papillomaviruses. Examples of pseudoviruses useful for practicing methods of the invention include, but are not limited to, a hepatitis pseudovirus, an influenza pseudovirus, and a papilloma pseudovirus. Thus, this disclosure provides a method of treating a hematologic cancer in an individual, comprising recruiting a preexisting immune response to the solid tumor by introducing to the tumor a recombinant virus, or pseudovirus, comprising a nucleic acid molecule encoding an antigenic protein, wherein the antigenic protein is recognized by one or more components of the preexisting immune response, and wherein the antigenic protein is not present in, or on, the hematologic cancer cells prior to treatment.

Introduction of viruses or pseudoviruses comprising an antigen-encoding nucleic acid molecule to a cancer can be achieved using any suitable method known in the art. For example, a recombinant virus, or pseudovirus, comprising the antigen-encoding nucleic acid molecule, can be administered to the individual by a route that results in delivery of the recombinant virus, or pseudovirus, to the cancer. Examples of such routes include, but are not limited to, intravenous (IV) injection, intramuscular (IM) injection, intra-peritoneal (IP) injection, subcutaneous (SC) injection, and oral administration. Thus, this disclosure provides a method of treating a hematologic cancer in an individual, comprising administering to the individual a recombinant virus, or pseudovirus, comprising a nucleic acid molecule encoding an antigenic protein, wherein the antigenic protein is recognized by one or more components of the preexisting immune response, and wherein the antigenic protein is not present in, or on, the hematologic cancer cells prior to treatment. The recombinant virus, or pseudovirus, may be delivered using a method selected from the group consisting of IV injection, IM injection, IP injection, SC injection, and oral administration.

The methods disclosed herein use one or more antigens to recruit a preexisting immune response to a cancer. Any antigen can be used, as long as the antigen is recognized by one or more components of a preexisting immune response, and the antigen is not present in, or on, the cancer cells prior to treatment. Examples of useful antigens include, but are not limited to, viral and bacterial antigens. One example of a viral antigen useful for practicing methods of the invention is an antigen comprising at least one epitope from a cytomegalovirus protein. As used herein, an epitope is a cluster of amino acid residues that is recognized by the immune system, thereby eliciting an immune response. Such epitopes may consist of contiguous amino acids residues (i.e., amino acid residues that are adjacent to one another in the protein), or they may consist of non-contiguous amino acid residues (i.e., amino acid residues that are not adjacent to one another in the protein) but which are in close special proximity in the finally-folded protein. It is generally understood by those skilled in the art that epitopes require a minimum of six amino acid residues to be recognized by the immune system. Thus, methods of the invention may include the use of antigens comprising at least one epitope from a cytomegalovirus protein. Any suitable CMV protein can be used to produce antigens useful for practicing methods of the invention, as long as the antigen recruits a preexisting immune response to a cancer. Examples of CMV proteins suitable for use in the methods disclosed herein include, but are not limited to, CMV pp50, CMV pp65, CMV pp150, CMV IE-1, CMV IE-2, CMV gB, CMV US2, CMV UL16, and CMV UL18. Examples of such protein, and useful fragments thereof, are disclosed in U.S. Patent Publication Nos. 2005/00193344 and 2010/0183647, both of which are incorporated herein by reference in their entirety. Useful fragments may also include any one or a combination of peptides comprising the amino acid sequence of SEQ ID NOS: 1-67.

The disclosed methods can also be practiced using one or more antigens, each of which independently comprises an amino acid sequence that is a variant of an at least 8 contiguous amino acid sequence from a CMV protein. As used herein, a variant refers to a protein, or nucleic acid molecule, the sequence of which is similar, but not identical to, a reference sequence, wherein the activity (e.g., immunogenicity) of the variant protein (or the protein encoded by the variant nucleic acid molecule) is not significantly altered. These variations in sequence can be naturally occurring variations or they can be engineered using genetic engineering techniques known to those skilled in the art. Examples of such techniques are found in Sambrook J, Fritsch E F, Maniatis T et al., in Molecular Cloning-A Laboratory Manual, 2nd Edition, Cold Spring Harbor Laboratory Press, 1989, pp. 9.31-9.57), or in Current Protocols in Molecular Biology, John Wiley & Sons, N.Y. (1989), 6.3.1-6.3.6. Regarding variants, any type of alteration in the amino acid sequence is permissible so long as the resulting variant protein retains the ability to elicit an immune response. Examples of such variations include, but are not limited to, deletions, insertions, substitutions and combinations thereof. For example, with proteins it is well understood by those skilled in the art that one or more (e.g., 2, 3, 4, 5, 6, 7, 8, 9 or 10), amino acids can often be removed from the amino and/or carboxy terminal ends of a protein without significantly affecting the activity of that protein. Similarly, one or more (e.g., 2, 3, 4, 5, 6, 7, 8, 9 or 10) amino acids can often be inserted into a protein without significantly affecting the activity of the protein.

As noted, variant proteins can contain amino acid substitutions relative to a reference protein (e.g., wild-type protein). Any amino acid substitution is permissible so long as the activity of the protein is not significantly affected. In this regard, it is appreciated in the art that amino acids can be classified based on their physical properties. Examples of such groups include, but are not limited to, charged amino acids, uncharged amino acids, polar uncharged amino acids, and hydrophobic amino acids. Preferred variants that contain substitutions are those in which an amino acid is substituted with an amino acid from the same group. Such substitutions are referred to as conservative substitutions.

Naturally occurring residues may be divided into classes based on common side chain properties: 1) hydrophobic: Met, Ala, Val, Leu, Ile; 2) neutral hydrophilic: Cys, Ser, Thr; 3) acidic: Asp, Glu; 4) basic: Asn, Gln, His, Lys, Arg; 5) residues that influence chain orientation: Gly, Pro; and 6) aromatic: Trp, Tyr, Phe.

For example, non-conservative substitutions may involve the exchange of a member of one of these classes for a member from another class.

In making amino acid changes, the hydropathic index of amino acids may be considered. Each amino acid has been assigned a hydropathic index based on its hydrophobicity and charge characteristics. The hydropathic indices are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5). The importance of the hydropathic amino acid index in conferring interactive biological function on a protein is generally understood in the art (Kyte et al., 1982, J. Mol. Biol. 157:105-31). It is known that certain amino acids may be substituted for other amino acids having a similar hydropathic index or score and still retain a similar biological activity. In making changes based upon the hydropathic index, the substitution of amino acids whose hydropathic indices are within ±2 is preferred, those within ±1 are particularly preferred, and those within ±0.5 are even more particularly preferred.

It is also understood in the art that the substitution of like amino acids can be made effectively based on hydrophilicity, particularly where the biologically functionally equivalent protein or peptide thereby created is intended for use in immunological invention, as in the present case. The greatest local average hydrophilicity of a protein, as governed by the hydrophilicity of its adjacent amino acids, correlates with its immunogenicity and antigenicity, i.e., with a biological property of the protein. The following hydrophilicity values have been assigned to these amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); and tryptophan (−3.4). In making changes based upon similar hydrophilicity values, the substitution of amino acids whose hydrophilicity values are within ±2 is preferred, those within ±1 are particularly preferred, and those within ±0.5 are even more particularly preferred. One may also identify epitopes from primary amino acid sequences based on hydrophilicity.

Desired amino acid substitutions (whether conservative or non-conservative) can be determined by those skilled in the art at the time such substitutions are desired. For example, amino acid substitutions can be used to identify important residues of the protein, or to increase or decrease the immunogenicity, solubility or stability of the protein. Exemplary amino acid substitutions are shown in the following table:

Amino Acid Substitutions

| Original Amino Acid | Exemplary Substitutions |
|---|---|
| Ala | Val, Leu, Ile |
| Arg | Lys, Gln, Asn |
| Asn | Gln |
| Asp | Glu |
| Cys | Ser, Ala |
| Gln | Asn |
| Glu | Asp |
| Gly | Pro, Ala |
| His | Asn, Gln, Lys, Arg |
| Ile | Leu, Val, Met, Ala |
| Leu | Ile, Val, Met, Ala |
| Lys | Arg, Gln, Asn |
| Met | Leu, Phe, Ile |
| Phe | Leu, Val, Ile, Ala, Tyr |
| Pro | Ala |
| Ser | Thr, Ala, Cys |
| Thr | Ser |
| Trp | Tyr, Phe |
| Tyr | Trp, Phe, Thr, Ser |
| Val | Ile, Met, Leu, Phe, Ala |

As used herein, the phrase “significantly affects a proteins activity” refers to a decrease in the activity of a protein by at least 10%, at least 20%, at least 30%, at least 40% or at least 50%. With regard to the present invention, such an activity may be measured, for example, as the ability of a protein to elicit neutralizing antibodies, or to elicit a T-cell response. Methods of determining such activities are known to those skilled in the art.

Methods of this disclosure may use one or more antigens, each of which independently comprises at least 6 contiguous amino acids, at least 10 contiguous amino acids, at least 20 contiguous amino acids, at least 30 contiguous amino acids, at least 50 contiguous amino acids, at least 75 contiguous amino acids, or at least 100 contiguous amino acids, from a CMV protein. Methods of this disclosure may use one or more antigens, each of which independently comprises an amino acid sequence at least 85% identical, at least 95% identical, at least 97% identical, or at least 99% identical, to at least 10 contiguous amino acids, at least 20 contiguous amino acids, at least 30 contiguous amino acids, at least 50 contiguous amino acids, at least 75 contiguous amino acids, or at least 100 contiguous amino acids, from a CMV protein. Methods of this disclosure may use one or more antigens, each of which independently comprises at least 6 contiguous amino acids, at least 10 contiguous amino acids, at least 20 contiguous amino acids, at least 30 contiguous amino acids, at least 50 contiguous amino acids, at least 75 contiguous amino acids, or at least 100 contiguous amino acids, from a CMV protein. Methods of this disclosure may use one or more antigens, each of which independently comprises an amino acid sequence at least 95% identical, at least 97% identical, or at least 99% identical, to 9 to 15 contiguous amino acid residues from a CMV protein, wherein the antigen is an MHC I-restricted antigen. Methods of this disclosure may use one or more antigens, each of which independently comprises 9 to 15 contiguous amino acid residues from a CMV protein, wherein the antigen is an MHC I-restricted antigen. Methods of this disclosure may use one or more antigens comprising an amino acid sequence at least 95% identical, at least 97% identical, or at least 99% identical, to at least 15 contiguous amino acid residues from a CMV protein, wherein the antigen is an MHC II-restricted antigen. Methods of this disclosure may use one or more antigens comprising at least 15 contiguous amino acid residues from a CMV protein, wherein the antigen is an MHC II-restricted antigen. Methods of this disclosure may one or more antigens comprising an amino acid sequence at least 95% identical, at least 97% identical, or at least 99% identical, to a peptide consisting of a sequence selected from the group consisting of peptides comprising the amino acid sequence of SEQ ID NOS: 1-67, or any combination thereof. Methods of this disclosure may use one or more antigens consisting of an amino acid sequence at least 95% identical, at least 97% identical, or at least 99% identical, to a sequence selected from the group consisting of peptides comprising the amino acid sequence of SEQ ID NOS: 1-67, or any combination thereof. Methods of this disclosure may use one or more antigens consisting of a sequence selected from the group consisting of peptides comprising the amino acid sequence of SEQ ID NOS: 1-67, or any combination thereof.

| SEQ ID NO | Amino Acid Sequence |
|---|---|
| 1 | LLQTGIHVRVSQPSL |
| 2 | PLKMLNIPSINVHHY |
| 3 | TRQQNQWKEPDVYYT |
| 4 | EPDVYYTSAFVFPTK |
| 5 | KVYLESFCEDVPSGK |
| 6 | TLGSDVEEDLTMTRN |
| 7 | QPFMRPHERNGFTVL |
| 8 | IIKPGKISHIMLDVA |
| 9 | EHPTFTSQYRIQGKL |
| 10 | YRIQGKLEYRHTWDR |
| 11 | TERKTPRVTGGGAMA |
| 12 | ASTSAGRKRKSASSA |
| 13 | ACTSGVMTRGRLKAE |
| 14 | AGILARNLVPMVATV |
| 15 | KYQEFFWDANDIYRI |
| 16 | PDDYSNTHSTRYVTV |
| 17 | HSRSGSVSQRVTSSQ |
| 18 | FETTGGLVVFWQGIK |
| 19 | YEYVDYLFKRMID |
| 20 | RSYAYIYTTYLLGSNTEYVA |
| 21 | NASYFGENADKFFIFPNYTI |
| 22 | LTFWEASERTIRSEAEDSYH |
| 23 | IRSEAEDSYHFSSAKMTATF |
| 24 | NEQAYQMLLALARLDAEQRA |
| 25 | YRNIEFFTKNSAFPKTTNG |
| 26 | FPKTTNGCSQAMAALQNLP |
| 27 | ARAKKDELRRKMMYMCYRN |
| 28 | SVMKRRIEEICMKVFAQYI |
| 29 | LVKQIKVRVDMVRHRIKEH |
| 30 | VKSEPVSEIEEVAPE |
| 31 | RRKMMYMCYRNIEFFTKNS |
| 32 | QLNRHSYLKDSDFLDAALDF |
| 33 | QGDKYESWLRPLVNVTRRDG |
| 34 | NLVPMVATV |
| 35 | FPTKDVAL |
| 36 | VTEHDTLLY |
| 37 | ELKRKMMYM |
| 38 | VLEETSVML |
| 39 | AYAQKIFKIL |
| 40 | IMREFNSYK |
| 41 | QYDPVAALF |
| 42 | DIYRIFAEL |
| 43 | TPRVTGGGAM |
| 44 | QIKVRVDMV |
| 45 | YSEHPTFTSQY |
| 46 | FEQPTETPP |
| 47 | ARVYEIKCR |
| 48 | QMWQARLTV |
| 49 | PFTSQYRIQGKL |
| 50 | CPSQEPMSIYVY |
| 51 | TRATKMQVI |
| 52 | ERAWALKNPH |
| 53 | GPISGHVLK |
| 54 | DALPGPCI |
| 55 | KMQVIGDQY |
| 56 | CEDVPSGKL |
| 57 | LYLCCGITL |
| 58 | VYVTVDCNL |
| 59 | LYTSRMVTNL |
| 60 | IPSINVHHY |
| 61 | QAIRETVEL |
| 62 | PGKISHIML |
| 53 | YEQHKITSY |
| 64 | TENGSFVAGY |
| 65 | QEFFWDANDI |
| 66 | YRNMIIHA |
| 67 | YAYIYTTYL |

Methods of the invention comprise treating an individual for cancer by recruiting a preexisting immune response to the cancer. In these methods, the individual may be known to have a preexisting immune response to an antigen, prior to initiation of the cancer treatment. The individual may be tested to confirm the presence of a preexisting immune response prior to initiating the cancer treatment. Thus, these methods may include treating cancer in an individual by confirming that the individual has a preexisting immune response to an antigen, wherein the antigen is not present in, or on, the cancer. The antigen is then administered to the individual confirmed to have the preexisting immunity, such that the antigen is introduced to the cancer, thereby treating the cancer.

Such a method can be used to treat any of the cancers already described herein, including any solid tumors and/or hematologic cancers.

Any method of confirming that the individual to be treated has a preexisting immune response to an antigen can be used to practice methods of this disclosure. Examples of such methods include identifying in a sample from the individual a B-cell that recognizes a specific antigen, an antibody that recognizes a specific antigen, a T-cell that recognizes a specific antigen, or T-cell activity that is initiated in response to a specific antigen. Any suitable sample from the individual can be used to identify a preexisting immune response. Examples of suitable samples include, but are not limited to, whole blood, serum, plasma, and tissue samples. As used herein, recognition of a specific antigen by a B-cell, T-cell, or an antibody, refers to the ability of such B-cells, T-cells, or antibodies to specifically bind the antigen. Specific binding of an antigen by a B-cell, T-cell, or antibody, means a B-cell, T-cell, or antibody, binds to a specific antigen with an affinity greater than the binding affinity of the same B-cell, T-cell, or antibody, for a molecule unrelated to the antigen. For example, a B-cell, T-cell, or antibody, that recognizes, or is specific for, an antigen from a CMV pp50 protein, binds the CMV pp50 antigen with an affinity significantly greater than the binding affinity of the same B-cell, T-cell, or antibody, for a protein unrelated to CMV pp50 protein, such as human albumin. Specific binding between two entities can be scientifically represented by their dissociation constant, which is often less than about 10⁻⁶, less than about 10⁻⁷, or less than about 10⁻⁸ M. The concept of specific binding, and methods of measuring such binding, between molecules, and cells and molecules, are well known to a person of ordinary skill in the art including, but not limited to, enzyme immunoassays (e.g., ELISA), immunoprecipitations, immunoblot assays and other immunoassays as described, for example, in Sambrook et al., supra, and Harlow et al., Antibodies, a Laboratory Manual (Cold Spring Harbor Labs Press, 1988). Such methods are also described in U.S. Pat. No. 7,172,873, which is incorporated herein by reference. Methods of measuring T-cell activation in a sample from an individual are also known to those skilled in the art. Examples of such methods are disclosed in U.S. Patent Publication No. 2003/003485, and in U.S. Pat. No. 5,750,356, both of which are incorporated herein by reference.

Such methods generally comprise contacting a T-cell containing sample from the individual with an antigen, and measuring the sample for activation of T-cells. Methods of measuring T-cell activation are also well known in the art and are also disclosed in Walker, S., et al., Transplant Infectious Disease, 2007:9:165-70; and Kotton, C. N. et al. (2013) Transplantation 96, 333.

Commercially available testing for CMV (QuantiFERON™-CMV, QIAGEN Sciences Inc., Germantown, MD) is available as an in vitro diagnostic test using a peptide cocktail simulating human cytomegalovirus proteins (CMV) to stimulate cells in heparinised whole blood. Individuals exposed to disease/infection have specific T cell lymphocytes in their blood that maintain an immunological memory for the antigens (immunologically reactive molecules) of the priming disease/infection. The addition of antigen to blood collected from a primed individual results in the rapid restimulation of antigen-specific effector T cells, resulting in the release of cytokines (e.g., IFN-γ). Effector T cells are able to respond quickly when exposed to the priming antigen. Thus, the production of IFN-γ in response to antigen exposure is a specific marker for cellular immune response against that antigen. This IFN-γ response may be used to quantify the immune response. Detection of interferon-gamma (IFN-γ) by Enzyme-Linked Immunosorbent Assay (ELISA) is used to identify in vitro responses to peptide antigens that are associated with CMV infection. The intended use of QuantiFERON™-CMV is to monitor the level of anti-CMV immunity in persons.

Thus, in any of the methods of this disclosure for treating cancer in an individual, the individual may first be confirmed to have a preexisting immune response to an antigen that is not present in, or on, the cancer. This preexisting immune response can be confirmed by identifying in a sample from the individual:
i) a B-cell that recognizes a specific antigen;
ii) an antibody that recognizes a specific antigen;
iii) a T-cell that recognizes a specific antigen; and,
iv) T-cell activity that is initiated in response to a specific antigen.
   The specific antigen may then be administered to the individual that is confirmed to have the preexisting immune response, such that the antigen is introduced to the cancer, thereby treating the cancer.

In any of the methods provided in this disclosure, other agents may be used (i.e., administered) in combination with the CMV antigens, within the practice of the current invention to augment the immune modulatory or recruitment. Such other agents which include, a TLR agonist; intravenous immunoglobulin (IVIG); peptidoglycan isolated from gram positive bacteria; lipoteichoic acid isolated from gram positive bacteria; lipoprotein isolated from gram positive bacteria; lipoarabinomannan isolated from mycobacteria, zymosan isolated from yeast cell wall; polyadenylic-polyuridylic acid; poly (IC); lipopolysaccharide; monophosphoryl lipid A; flagellin; Gardiquimod; Imiquimod; R848; oligonucleosides containing CpG motifs, a CD40 agonist, and 23S ribosomal RNA. In a preferred aspect of these methods, the TLR agonist is poly-IC.

Another aspect of this disclosure are kits for testing an individual and recruiting a preexisting immune response to a cancer in the individual. The kit may comprise at least one CMV peptide antigen or a nucleic acid encoding the peptide, a pharmaceutically acceptable carrier, a container, and a package insert or label indicating the administration of the CMV peptide for reducing at least one symptom of the cancer in the patient. These kits may further include means for testing the patient's antigenic response to CMV antigens. For example, the kit may include sterilized plasticware for obtaining and testing a whole blood sample, and in vitro testing of responses to CMV peptide antigens and/or detection of interferon-gamma (IFN-γ) by Enzyme-Linked Immunosorbent Assay (ELISA) to identify in vitro responses to these peptide antigens.

### EXAMPLES

Chronic viral infections that are normally well controlled by the host, for example human Cytomegalovirus (hCMV), often lead to the induction of increasingly large numbers of fully functional virus-specific T cells with age. Using a mouse mCMV model that mimics critical aspects of the human immune response to hCMV, the inventors have developed methods and reagents to attract these antiviral T cells to tumors, with subsequent killing of the tumor cells and induction of potent epitope spreading to tumor neoantigens that results in adaptive immune responses conferring long term control of tumor growth and protection from rechallenge with homologous tumor cells.

### Example 1

### Murine Cytomegalovirus Infection Induces Cytokine Response Against mCMV Peptide Pool

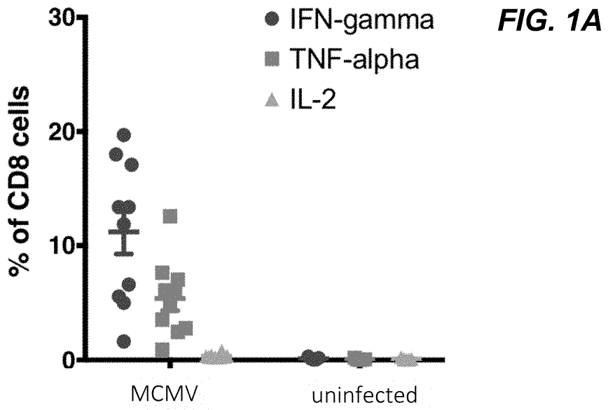

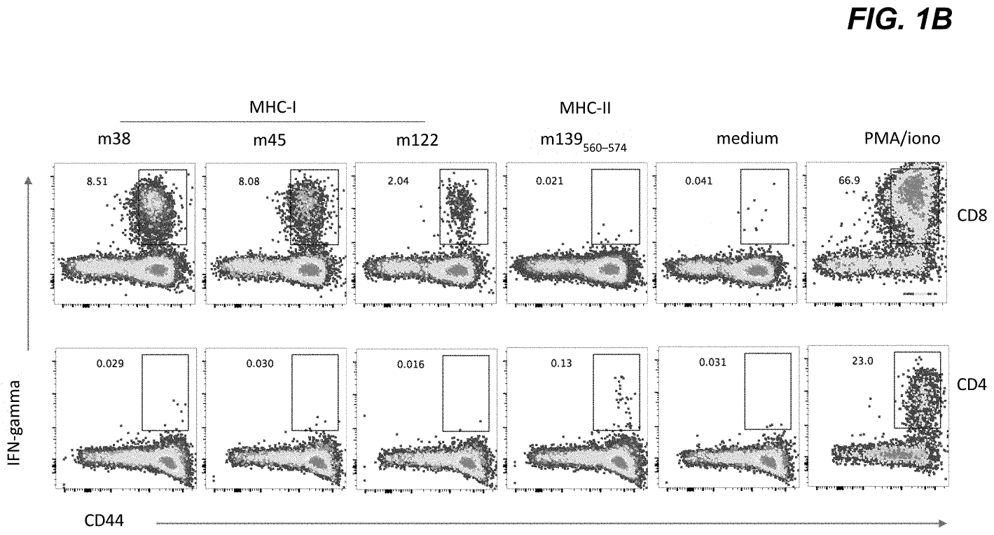

C57Bl/6 mice were infected with 1×10\hat{ }4 pfu murine cytomegalovirus (mCMV). Blood samples were collected on day 12 post infection. Blood leukocytes were re-stimulated with a pool of selected immunogenic peptides from m38, m45, m57, m122, 1m39, m141, and m164 mCMV proteins. IFN-gamma, TNF-alpha, and IL-2 cytokines production by CD8+ T cells was assessed by intracellular cytokine staining and analyzed by fluorescence-activated cell sorting (FACS) (FIG. 1A). Blood samples were collected two months after infection. Inflationary (m122) and non-inflationary (m45) specific CD8+ T cells were detected by FACS using MHC-I tetramer staining. Memory CD8+ T cell responses were mapped against mCMV. Spleens were collected six months after infection. IFN-gamma production by CD8+ and CD4+ T cells after in vitro stimulation with m38, m45, m122 MHC-I restricted and m139560-574 MHC-II restricted mCMV peptide was assessed by intracellular cytokine staining (FIG. 1B).

### Example 2

### Intratumoral Transduction of Solid Tumors with HPV Psv Expressing mCMV Antigens

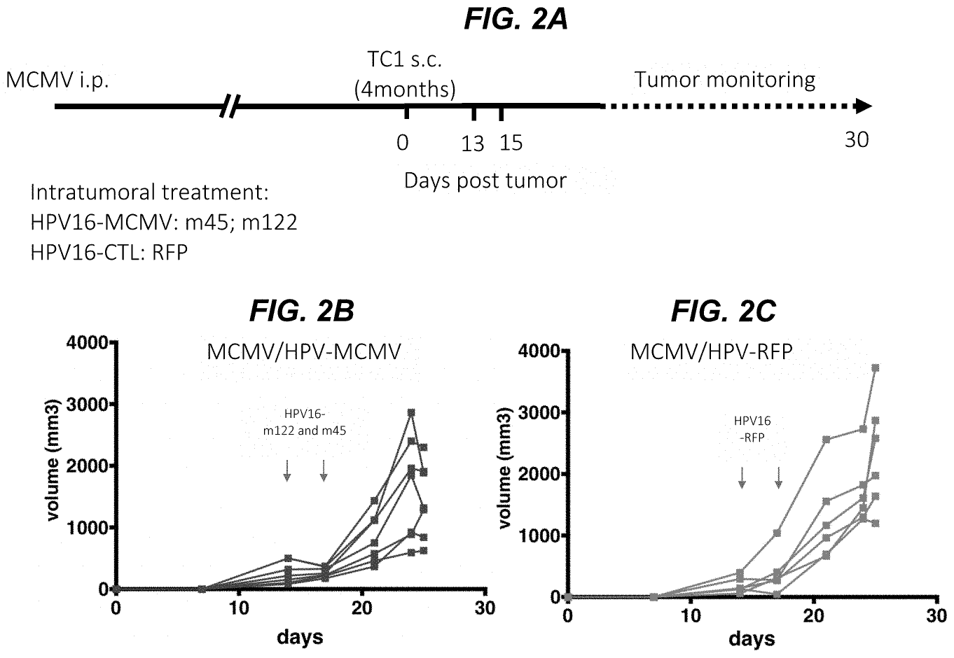

C57Bl/6 mice were infected with 1×10\hat{ }4 pfu murine cytomegalovirus (mCMV). Six months after infection, mice were injected s.c. with 2×10\hat{ }5 TC-1 tumor cells expressing E6 an E7 oncoproteins (injection protocol, FIG. 2A). Tumor growth was measured using an electronic caliper. On day 13 and day 15 after tumor injection, HPV16 Psv expressing m122 and m45 (FIG. 2B), or HPV Psv expressing red fluorescent protein (RFP) (FIG. 2C) were injected intratumoral (10\hat{ }8 infectious units per PsV).

### Example 3

### Intratumoral Transduction of Solid Tumors with mCMV Antigens

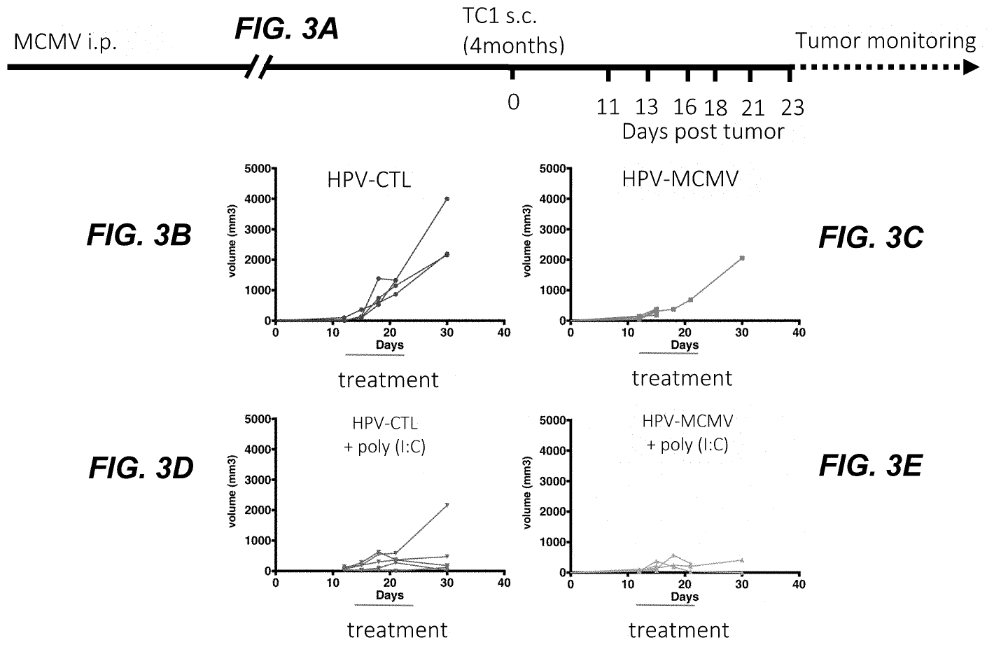

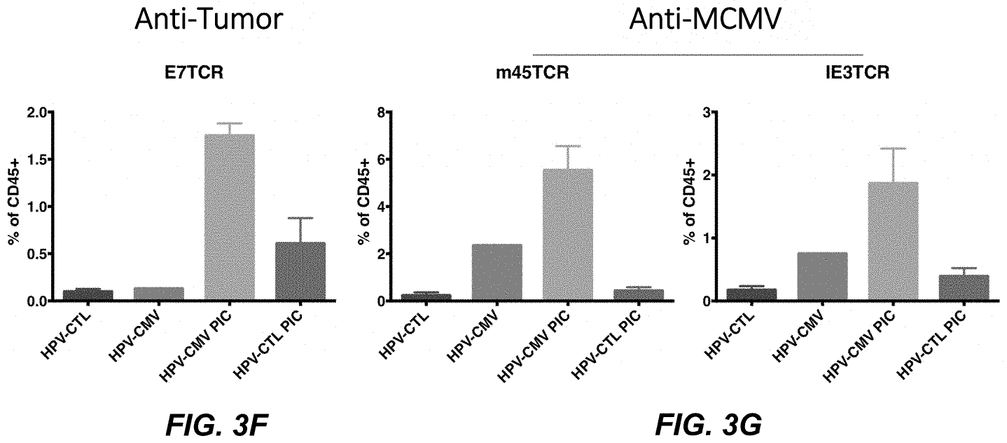

Combined with Poly(I:C) C57Bl/6 mice were infected with 1×10\hat{ }4 pfu murine cytomegalovirus (mCMV). Four months after infection, mice were injected s.c. with 2×10\hat{ }5 TC-1 tumor cells expressing E6 an E7 oncoproteins (FIG. 3A). Tumors were injected intratumoral on days 11 and 13 with HPV16, on days 16 and 18 with HPV45, and on days 21 and 23 with HPV58 expressing m122, m38 and m45, or control RFP (10\hat{ }8 infectious units per PsV) with or without poly(I:C) (30 μg) (PIC). Tumor growth was measured using an electronic caliper (FIGS. 3B-3E). These tumor volume/growth data demonstrate that the intratumoral transduction of solid tumors with HPV Psv expressing mCMV antigens slows tumor growth, and co-administration with poly(I:C) further slows tumor growth (compare FIGS. 3B and 3D; and compare FIGS. 3C and 3E). Infiltration of tumors by E7- (FIG. 3F), m45-, and m122- (FIG. 3G) specific CD8+ T cells was analyzed by MHC-I tetramer staining and FACS. These data demonstrate the significantly enhanced tumor infiltration of CD8+ T cells when these CMV antigens are administered in combination with poly(IC).

### Example 4

### Intratumoral Injection of mCMV MHC-I Restricted Peptides Confers Increased Survival

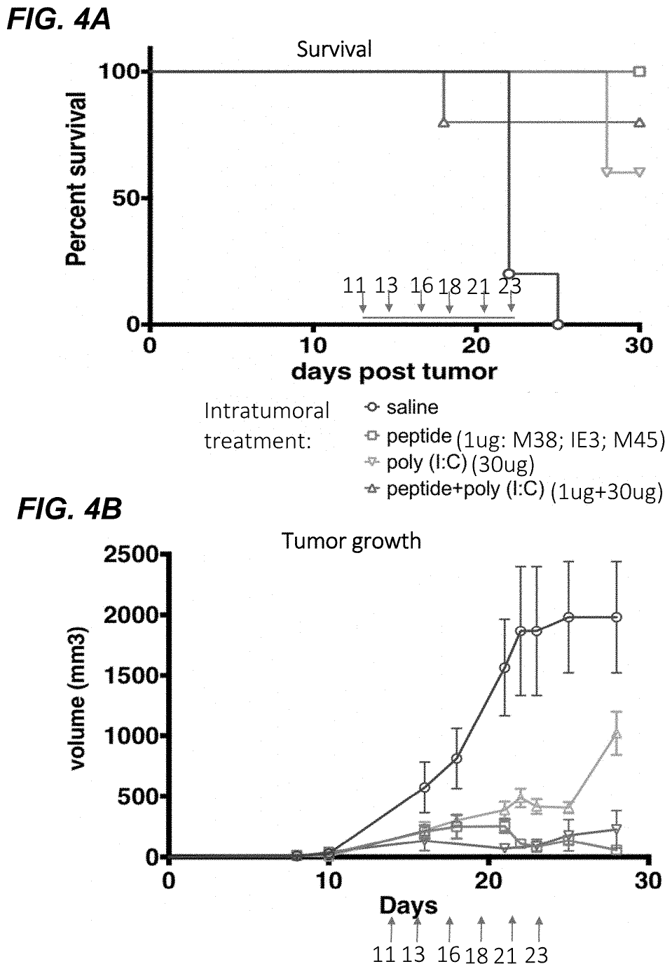

C57Bl/6 mice were infected with 1×10\hat{ }4 pfu murine cytomegalovirus (mCMV). Four months after infection, mice were injected s.c. with 2×10\hat{ }5 TC-1 tumor cells expressing E6 an E7 oncoproteins (FIG. 3A). Tumors were injected intratumoral on day 11, 13, 16, 18, 21, and 23 with selected m38, m45, and m122 peptides (1 μg each) with or without poly(I:C) (30 ug), and saline or poly(I:C) alone as controls. Animal deaths were recorded (FIG. 4A) and tumor growth was measured using an electronic caliper (FIG. 4B). These data demonstrate that intratumoral injection of mCMV MHC-I restricted peptides delays tumor growth and confers increased survival.

### Example 5

### Intratumoral Injection of mCMV MHC-I Restricted Peptides Delays Tumor Growth

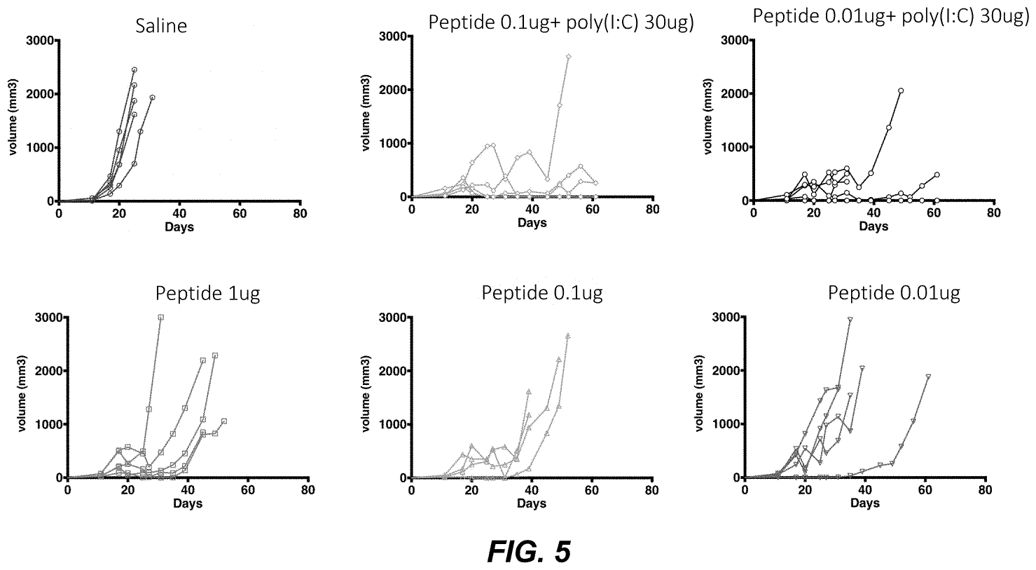

C57Bl/6 mice were infected with 1×10\hat{ }4 pfu murine cytomegalovirus (mCMV). Four months after infection, mice were injected s.c. with 2×10\hat{ }5 TC-1 tumor cells expressing E6 an E7 oncoproteins. Tumors were injected intratumoral on day 11, 13, 16, 18, 21 and 23 with decreasing doses (1 μg, 0.1 μg, and 0.01 μg) of selected m38, m45, and m122 peptide with or without poly(I:C) (30 ug), and saline or poly(I:C) alone as controls. Tumor growth was measured using an electronic caliper (FIG. 5). These data demonstrate that intratumoral injection of mCMV MHC-I restricted peptides delays tumor growth.

### Example 6

### Combinations of mCMV MHC-I and MHC-II Restricted Peptides Delays Tumor Growth

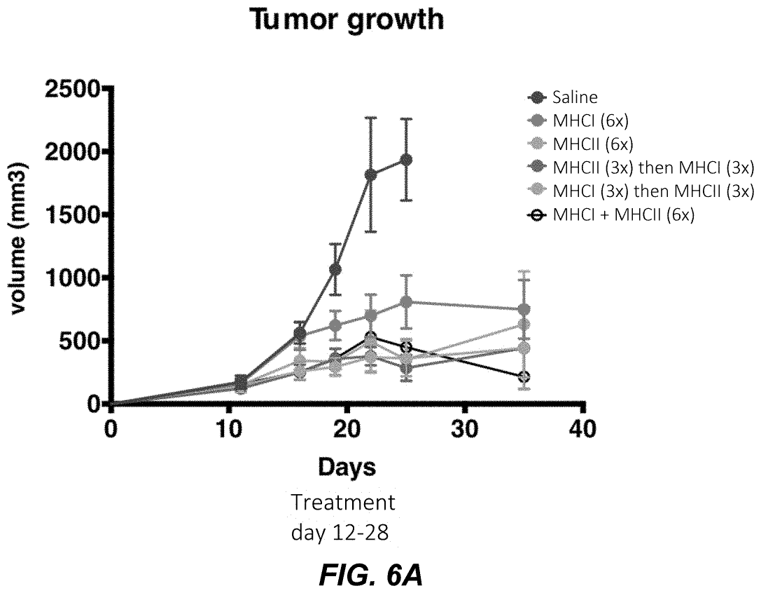

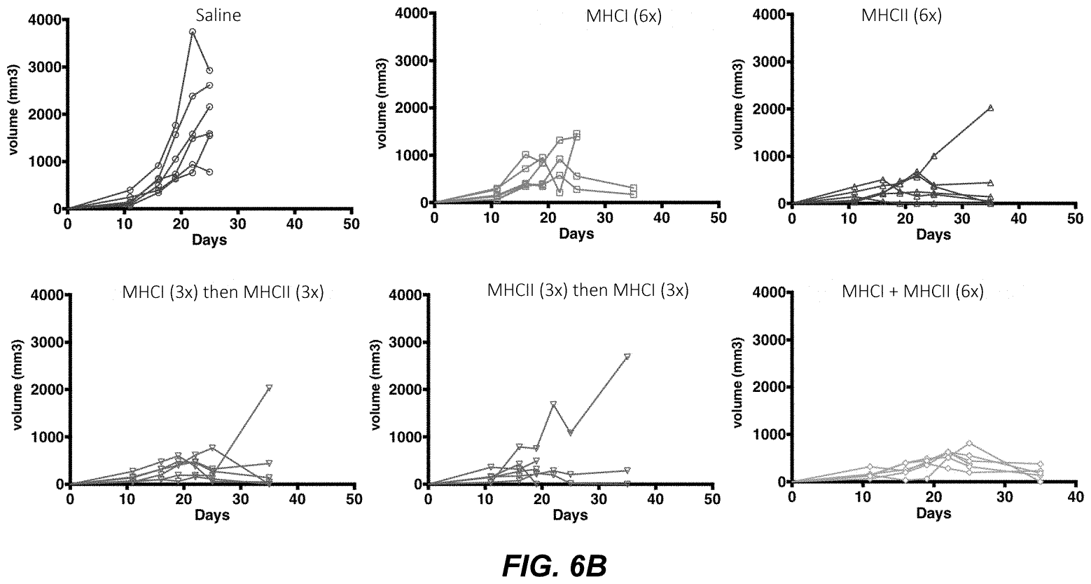

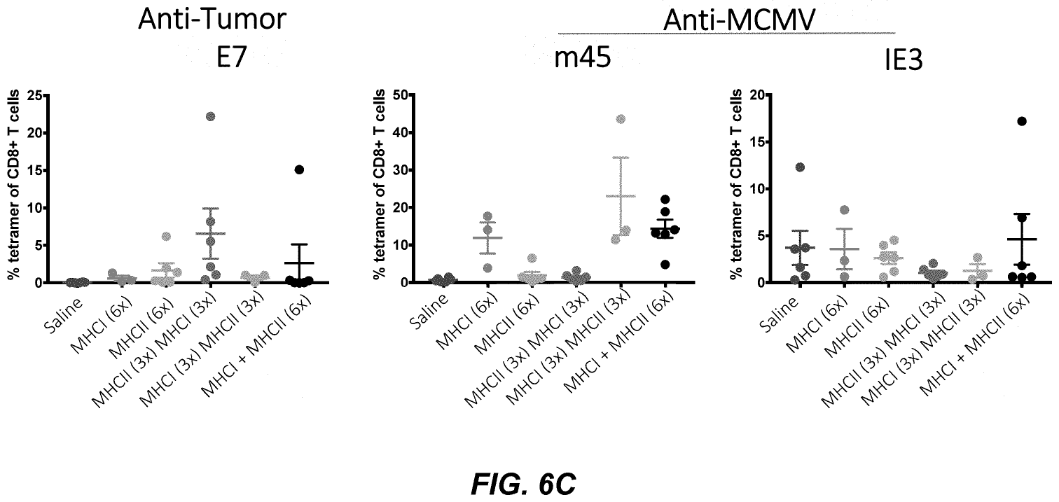

C57Bl/6 mice were infected with 2.5×10\hat{ }5 mCMV. Four months after infection, mice were injected s.c. with 2×10\hat{ }5 TC-1 tumor cells expressing E6 an E7 oncoproteins. Tumors were injected intratumoral 6 times from day 12 to day 28 with MHC-I restricted selected m38, m45 and m122 peptide, and/or MHC-II restricted m139 selected peptide or saline. All peptides were injected with poly(I:C) (30 μg). Groups were injected 6 times with MHC-I, or 6 times with MHC-II peptides, or 6 times with MHCI and MHCII peptides together, or sequentially 3 times with MHC-I peptides followed by 3 times MHC-II peptides, or 3 times with MHC-II peptides followed by 3 times with MHC-I peptides. Tumor growth was measured using an electronic caliper (FIGS. 6A and 6B). These data demonstrate that intratumoral injection of combinations of mCMV MHC-I and MHC-II restricted peptides delays tumor growth. E7-, m45-, m122-specific CD8+ T cell responses in blood were also analyzed by FACS using MHC-I tetramers for each peptide (FIG. 6C). These data demonstrate that sequential intratumoral inoculation with mCMV CD4 and then CD8 epitopes preferentially induces anti-tumor immunity.

### Example 7

### Complete Clearance of Primary Tumors Confers Long Term Tumor Protection

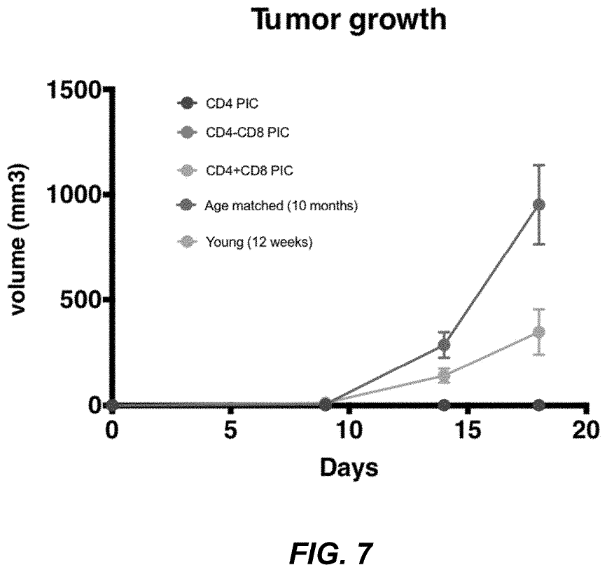

Protected C57Bl/6 mice that survived primary tumor challenge as described in Example 6 were injected s.c. with 2×10\hat{ }5 TC-1 tumor cells expressing E6 an E7 oncoproteins on the opposite flank of the primary challenge. As controls for tumor take, young (12 weeks old) and age matched (10 months old) mice were challenged with TC-1 tumor cells. Tumor growth was measured using an electronic caliper (FIG. 7). These data demonstrate that complete clearance of primary tumors confers long term protection against secondary tumor challenge.

### Example 8

### Intratumoral Injection of MCMV Alters the Tumor Immune Microenvironment

The effect of intratumoral injection of mCMV MHC-I and MHC-II restricted peptides, with or without polyIC, on the tumor immune microenvironment was analyzed in RNA samples for immune gene expression using Nanostring Cancer immunology gene set (nCounter), two days after the end of the last intratumoral treatment. Results were summarized by score change for each gene set analyzed. Global scores of differential expression by gene sets were made relative to saline-treated groups (n=4 per group). Microenvironment characteristics evaluated included: B-cell functions, Interleukins, TNF superfamily, Antigen processing, MHC, Adaptive, Transporter functions, Adhesion, NK cell functions, T-cell functions, CD molecules, Leukocytes functions, Complement pathway, Microglial function, Humoral, TLR, Inflammation, Dendritic cell functions, Interferon, Innate, Macrophages functions, Chemokines and receptors, Senescence, Apoptosis, Cytokines and receptors, Cancer progression, Basic cell functions, Cell cycle, and Pathogen response.

### Example 9

### mCMV Infection Induces an Inflationary CD8+ T Cell Response in C57BL/6 Mice

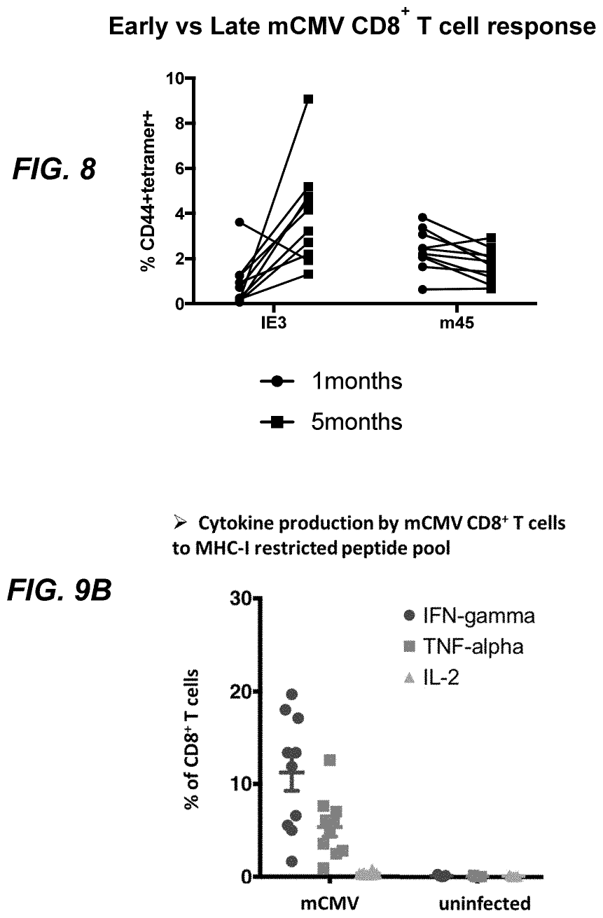

C57Bl/6 mice were infected with 5×10\hat{ }3 pfu murine cytomegalovirus (mCMV). Blood samples were collected 1 or 5 months after infection. Inflationary (IE3) and non-inflationary (m45) specific CD8+ T cells were detected by FACS using MHC-I tetramer staining. As shown in FIG. 8, mCMV infection induced distinct effector and memory CD8+ T cell responses.

### Example 10

### mCMV Infection Induces Potent CD8+ and Car T Cell Responses in C57BL/6 Mice

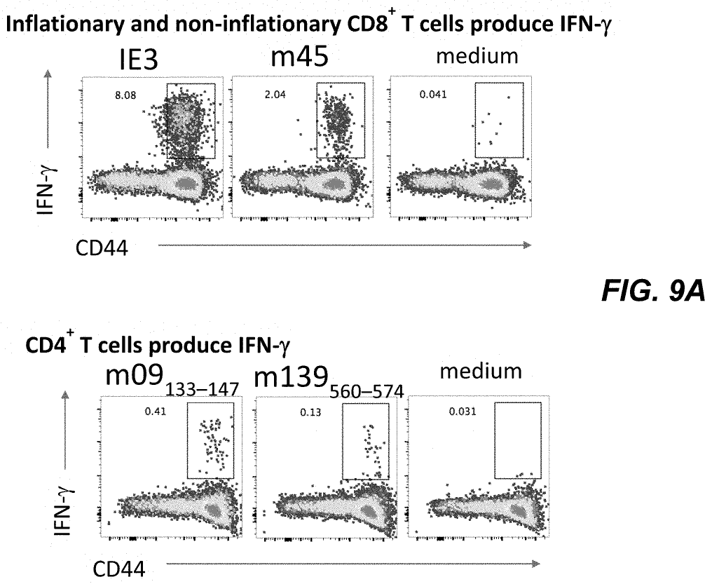

C57Bl/6 mice were infected with 5×10\hat{ }3 pfu murine cytomegalovirus (mCMV). Blood samples were collected on day 12 post infection. Spleen cells were re-stimulated with the indicated peptides and blood cells with a pool of selected immunogenic peptides from m38, m45, m57, m122, m139, m141, and m164 mCMV proteins. IFN-gamma, TNF-alpha, and IL-2 cytokine production by CD4+ and CD8+ T cells was assessed by intracellular cytokine staining and analyzed by FACS (FIGS. 9A, 9B). These results show that murine cytomegalovirus infection induces a massive cytokine response.

### Example 11

### Tissue Distribution of mCMV-Specific CD8+ T Cells

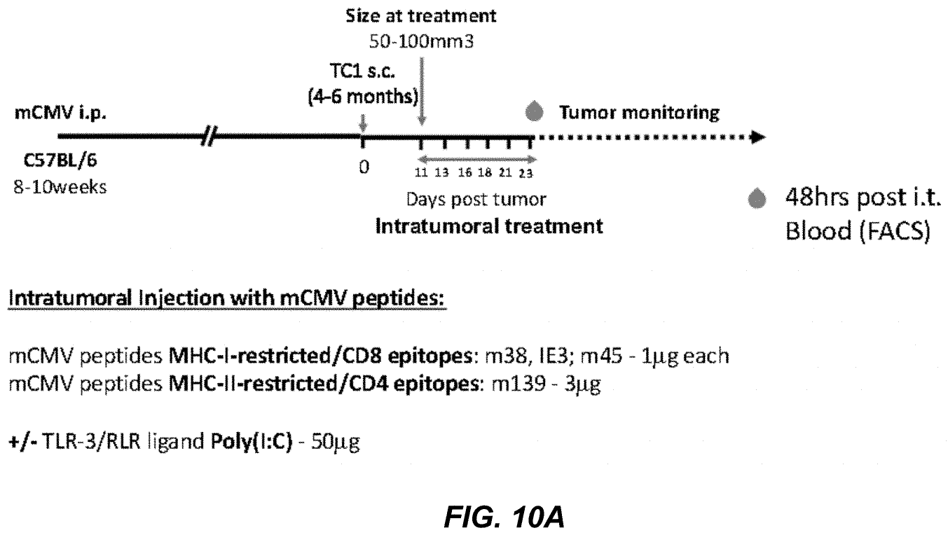

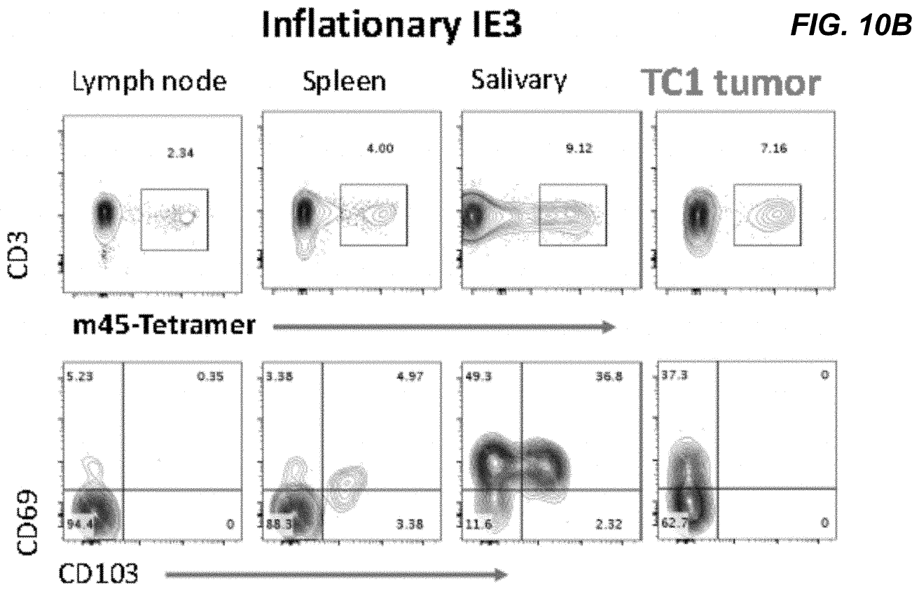

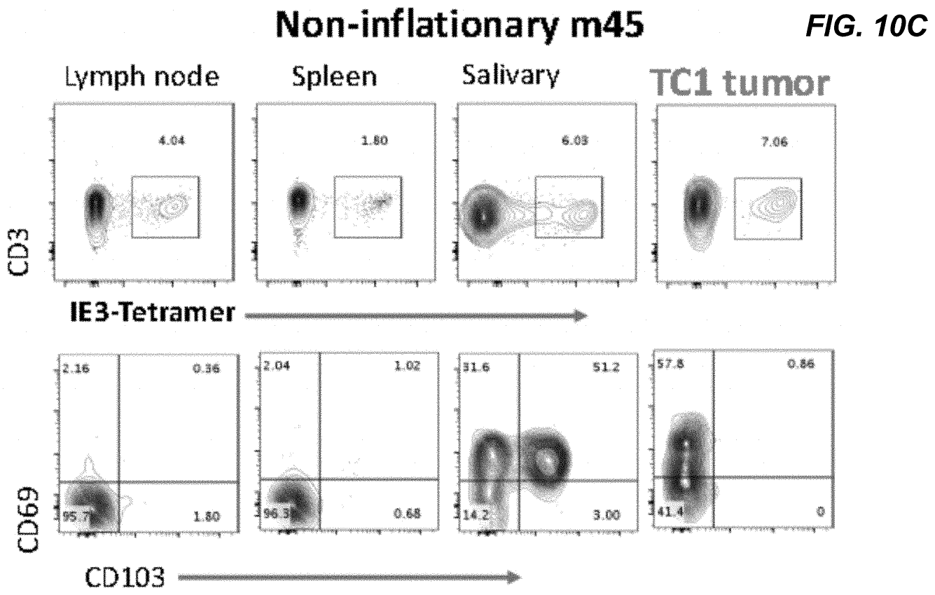

The distribution of mCMV-specific CD8+ T cells in tumor bearing mice was investigated. C57Bl/6 mice were infected with 5×10\hat{ }3 mCMV. The experimental schedule is shown in FIG. 10A. Four months after infection, mice were injected s.c. with 2×10\hat{ }5 TC-1 tumor cells expressing E6 an E7 oncoproteins. Lymph nodes, spleen, salivary glands and tumor tissues were collected and inflationary (IE3; FIG. 10B) and non-inflationary (m45; FIG. 10C) specific CD8+ T cells were detected by FACS using MHC-I tetramer staining. Expression of resident memory T cells marker was assessed using CD69 and CD103 antibodies. These results showed that TC1 tumors were infiltrated by mCMV-specific CD8+ T cells.

### Example 12

### Gene Expression Analysis of Tumor Microenvironment

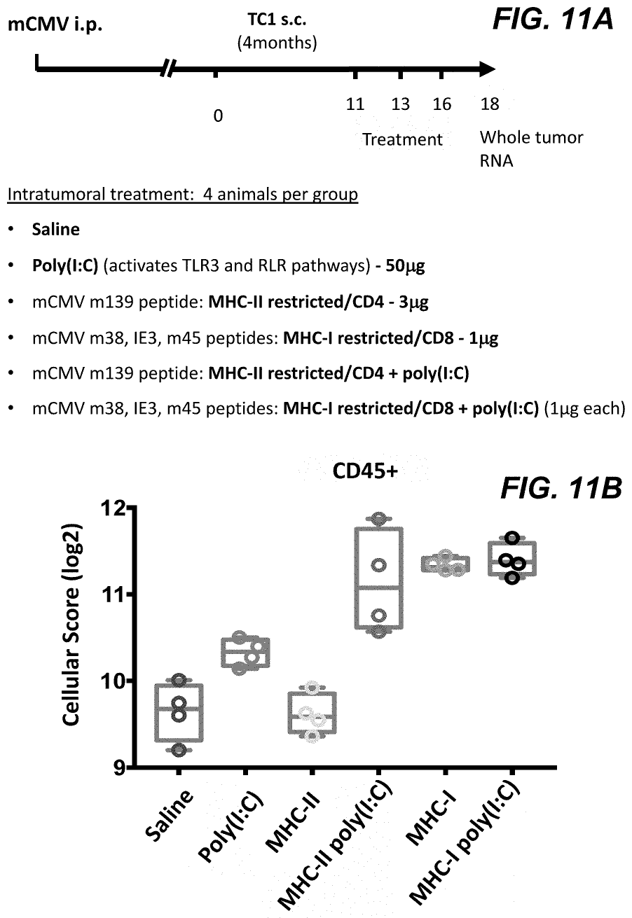

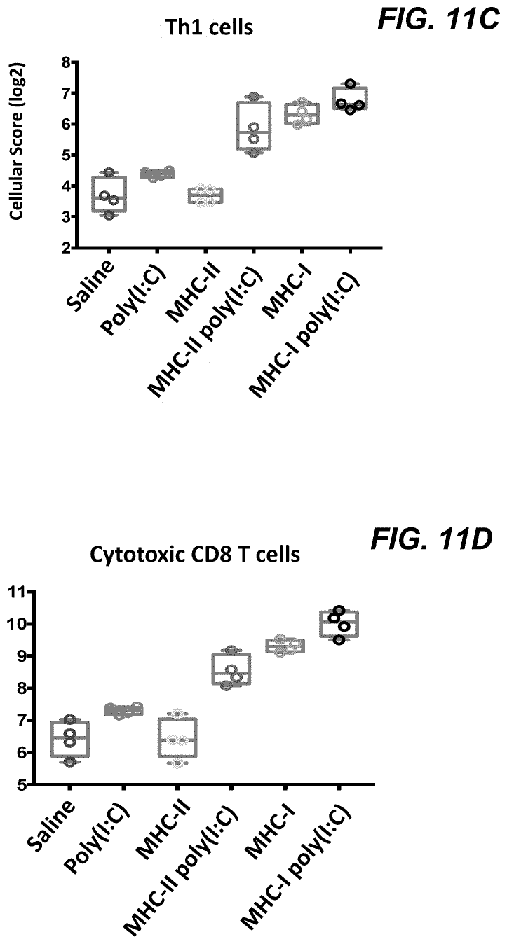

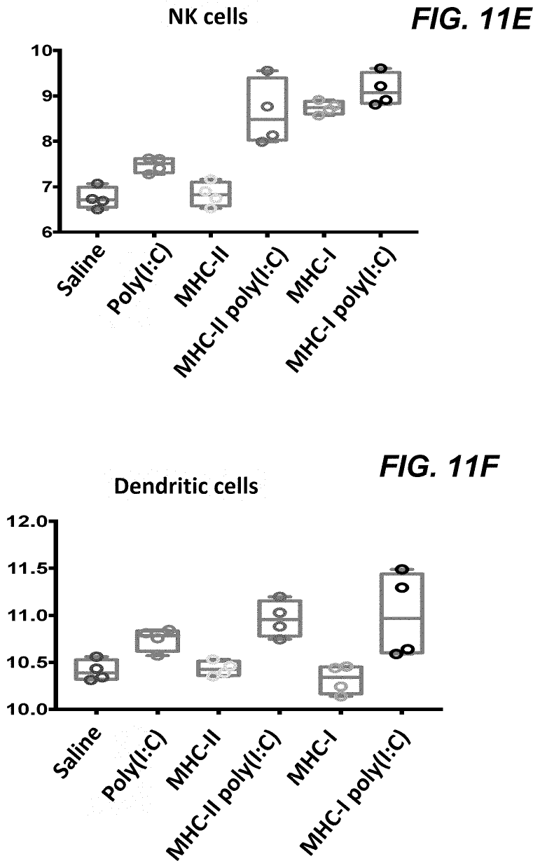

The expression of genes in tumor cells in the mouse model was investigated following intratumoral treatment (4 animals per group) with saline; PolyI:C (PIC) (50 μg); mCMV m139 peptide (MHC-II restricted/CD4) (CD4) (3 μg); mCMV m38, m122, m45 peptides (MHC-I restricted/CD8) (CD8) (1 μg each); mCMV m139+polyI:C (PIC CD4) (3 μg each); mCMV m38, m122, m45 peptides (MHC-I restricted/CD8)+polyI:C (PIC CD8) (1 μg each). Tumors were treated three times at 11, 13, and 16 weeks after TC1 tumor cells were placed subcutaneously. The experimental protocol timeline is shown in FIG. 11A. Following treatment and tumor harvest, tumor RNA was extracted using a QIACube. Tumor cell gene expression was analyzed using the Nanostring Cancer immunology gene set (NS_MM_CANCERIMM_C3400) which measures gene transcripts form 770 genes in the tumor PanCancer Immune Profiling Panel: Briefly, normalized data is represented as heat map of gene sets expression within a specific of biological processes (Adaptive immunity, antigen processing, T cell functions, dendritic cell functions, NK cell functions, Interferons, TNF superfamily genes); a Volcano Plot of gene expression changes relative to Saline treatment is constructed (the plot represents changes (expressed as fold-increase or -decrease) in treatment groups relative to control treatment (saline) with statistical significance); the cell infiltration quantification algorithm is applied (CD45, cytotoxic CD8, CD4 Th1, NK cells, and dendritic cells). The results showed the greatest change in global significance scores in the MHC-I restricted/CD8 and MHC-I restricted/CD8+poly(I:C) treated animals.

Profiling of immune genes in the whole tumor RNA after intratumoral treatment showed significant upregulation of immune genes in three groups:
1) mCMV m139 peptide: MHC-II restricted/CD4−3 mg (230 genes up-regulated, and 4 down regulated);
2) mCMV m38, IE3, m45 peptides: MHC-I restricted/CD8−1 mg (359 genes up-regulated, and 43 down regulated);
3) mCMV m38, IE3, m45 peptides: MHC-I restricted/CD8+poly(I:C) (309 genes up-regulated, and 49 down regulated).

The infiltration of the tumors by leucocytes was also analyzed after the intratumoral treatments. FIGS. 11B-11F show the tumor infiltration by different leucocytes. These data showed that intratumoral injection of CD8 mCMV epitopes (with or without poly(I:C)) induces the recruitment of T cells and non T cells (NK) in the tumor; and intratumoral injection of CD4 mCMV epitopes with poly(I:C) induces the recruitment of T cells and non T cells (NK) in the tumor; and poly(I:C) intratumoral injection with CD8 or CD4 epitopes induces the recruitment of dendritic cells in the tumor.

### Example 13

### Intratumoral Injection of mCMV CD8 Epitopes Delays Tumor Growth

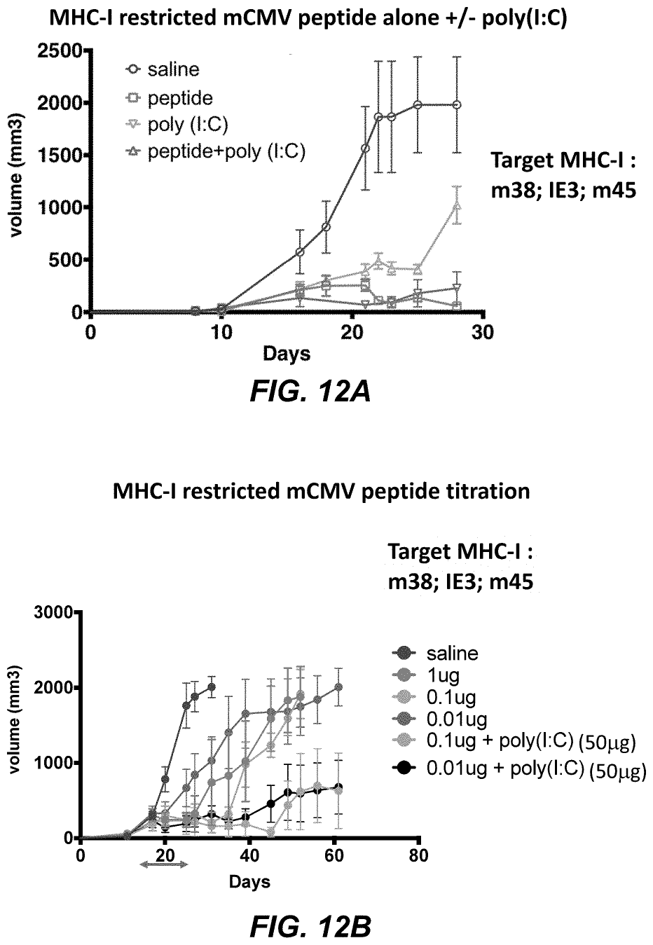

C57Bl/6 mice were infected with 5×10\hat{ }3 pfu murine cytomegalovirus (mCMV). Four months after infection, the mice were injected s.c. with 2×10\hat{ }5 TC-1 tumor cells expressing E6 an E7 oncoproteins. Tumor growth was measured using an electronic caliper. Tumors were injected intratumoral on day 11, 13, 16, 18, 21 and 23 with selected MHC-I restricted m38, m45 and m122 peptides (0.01, 0.1 or 1 μg each) with or without poly(I:C)(30 μg), and saline or poly(I:C) alone, as controls. FIGS. 12A and 12B show that intratumoral injection of mCMV MHC-I restricted peptides delays tumor growth, and poly(I:C) co-injection improves tumor control.

### Example 14

### Protection from TC1 and MC38 Tumor Challenge by Intratumoral Injection of mCMV MHC-I and/or MHC-II Peptides with Poly(I:C)

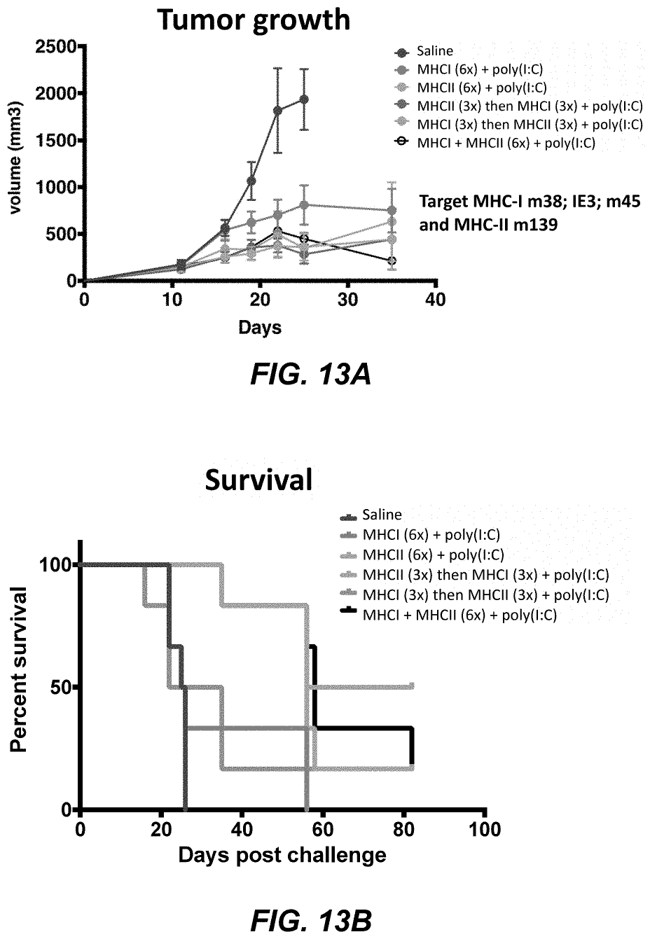

C57Bl/6 mice were infected with 5×10\hat{ }3 mCMV. Four months after infection, mice were injected s.c. with 2×10\hat{ }5 TC-1 tumor cells expressing E6 an E7 oncoproteins. Tumor growth and survival were monitored. Tumors were injected intratumoral 6 times from day 12 to day 28 with MHC-I restricted selected m38, m45, and m122 peptides, and/or MHC-II restricted m139 selected peptide with or without poly(I:C)(30 μg), and saline or poly(I:C) alone as controls. Groups were injected 6 times with MHC-I, or 6 times with MHC-II peptides, or 6 times with MHC-I and MHC-II peptides together, or sequentially 3 times with MHC-I peptides followed by 3 times MHC-II peptides, or 3 times with MHC-II peptides followed by 3 times with MHC-I peptides. FIG. 13A shows that intratumoral injection of combinations of mCMV MHC-I and MHC-II restricted peptides delays tumor growth, and

FIG. 13B shows sequential intratumoral inoculation with CD4 (MHC-II) then CD8 (MHC-I) mCMV epitopes promotes long-term survival.

### Example 15

### E7 Tetramer Positive CD8+ T Cell Responses in Blood after Treatments

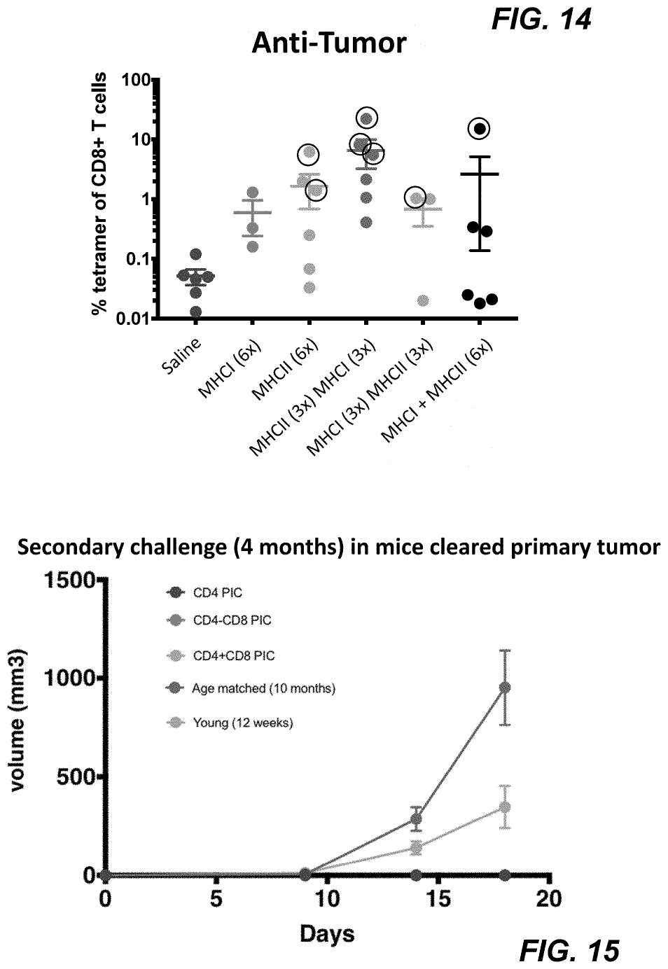

C57Bl/6 mice were infected with 5×10\hat{ }3 mCMV. Four months after infection, mice were injected s.c. with 2×10\hat{ }5 TC-1 tumor cells expressing E6 an E7 oncoproteins. Tumor size was measured using an electronic caliper. Tumors were injected intratumoral 6 times from day 12 to day 28 with MHC-I restricted selected m38, m45, and m122 peptide and/or MHC-II restricted m139 selected peptide with or without poly(I:C)(30 ug), and saline or poly(I:C) alone as controls. All peptides were injected with Poly(I:C)(30 ug). Groups were injected 6 times with MHC-I, or 6 times with MHC-II peptides, or 6 times with MHC-I and MHC-II peptides together, or sequentially 3 times with MHC-I peptides followed by 3 times MHC-II peptides, or 3 times with MHC-II peptides followed by 3 times with MHC-I peptides. E7-, m45-, m122-specific CD8+ T cell responses in blood were analyzed by FACS using MHC-I tetramers for each peptide. FIG. 14 shows that sequential intratumoral inoculation with mCMV CD4 then CD8 epitopes preferentially induces anti-tumor immunity.

### Example 16

### Long Term Protection Against Secondary Tumor Challenge

Protected C57Bl/6 mice which survived primary tumor challenge as described above were injected s.c. with 2×10\hat{ }5 TC-1 tumor cells expressing E6 an E7 oncoproteins on the opposite flank of the primary challenge. Tumor growth was measured using an electronic caliper. As controls for tumor take, young (12 weeks old) and age matched (10 months old) mice were challenged with TC-1 tumor cells. FIG. 15 shows that complete clearance of primary tumors confers long term protection against secondary tumor challenge.

### Example 17

### Protection from MC38 Tumor Challenge by Intratumoral Injection of mCMV MHC-I and MHC-II Peptides with Poly(I:C)

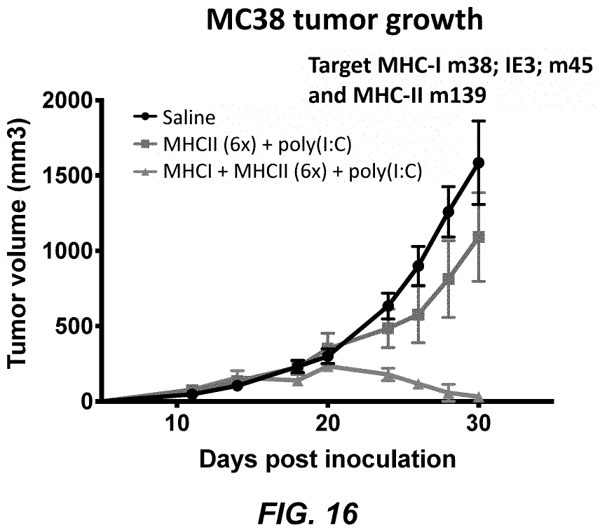

C57Bl/6 mice were infected with 5×10\hat{ }3 mCMV. Four months after infection, mice were injected s.c. with 5×10\hat{ }5 MC38 tumor cells from a mouse colon adenocarcinoma displaying hypermutation and microsatellite instability. Tumor growth was monitored. Tumors were injected intratumoral 6 times from day 12 to day 28 with MHC-I restricted selected m38, m45, and m122 peptides, and MHC-II restricted m139 selected peptide with poly(I:C)(30 μg), or MHC-II restricted m139 selected peptide alone with poly(I:C)(30 μg) and saline alone as control. FIG. 16 shows that complete clearance of primary tumors confers long term protection against secondary tumor challenge. FIG. 16 shows that intratumoral injection of combinations of mCMV MHC-I and MHC-II restricted peptides delays tumor growth and leads to tumor clearance.

The studies described in Examples 1-17 demonstrate that both non-inflationary and inflationary mCMV-specific T cells infiltrate tumors during latent mCMV infection, and redirecting established anti-viral T cells into solid tumor leads to tumor regression, to profound alteration in the tumor immune micro environment. The data also show that redirecting established anti-viral CD4+ T cells into solid tumor promotes epitope spreading to tumor-associated antigens and complete tumor clearance. These methods therefore provide broadly applicable “antigen agnostic” tumor therapies based on preexisting antiviral T cells. HPV L1 and L2 particles display strong tropism to numerous tumor cells but do not bind or infect intact epithelia. HPV PsV or VLP can therefore be used to direct anti-tumor agents genetically or directly as a carrier to tumor cells.

While the present invention has been described with reference to the specific embodiments, it should be understood by those skilled in the art that various changes may be made, and equivalents may be substituted, without departing from the true spirit and scope of the invention. In addition, many modifications may be made to adapt a particular situation, material, composition of matter, process, process step or steps, to the objective, spirit and scope of the present invention. All such modifications are intended to be within the scope of the claims.

1.-29. (canceled)

30. A method of treating a solid tumor in a subject in need thereof, comprising administering to the solid tumor at least one immunogenic MHC-II-restricted antigen from a human cytomegalovirus (CMV) protein, wherein each MHC-II-restricted antigen recruits a naturally-occurring preexisting immune response to CMV to the solid tumor, wherein the MHC-II-restricted antigen is not expressed by any of the cancer cells of the solid tumor, and wherein the antigen is recognized by one or more components of the preexisting immune response.

31. The method of claim 30, wherein the CMV protein is selected from the group consisting of pp50, pp65, pp150, gB, gL, gH, IE-1, IE-2, US2, US6, UL16, and UL18.

32. The method of claim 31, wherein the at least one immunogenic MHC-II-restricted antigen comprises the sequence set forth in one of SEQ ID NOs: 1-33.

33. The method of claim 32, comprising administering MHC-II-restricted antigens comprising the sequences set forth in SEQ ID NOs: 1-33, wherein each MHC-II-restricted antigen comprises the sequence set forth in one of SEQ ID NOs: 1-33.

34. The method of claim 30, further comprising administering an agent that augments the immune response.

35. The method of claim 34, wherein the agent that augments the immune response is selected from the group consisting of a TLR agonist; an IL-1R8 cytokine antagonist; intravenous immunoglobulin (WIG); peptidoglycan isolated from gram positive bacteria; lipoteichoic acid isolated from gram positive bacteria; lipoprotein isolated from gram positive bacteria; lipoarabinomannan isolated from mycobacteria; zymosan isolated from yeast cell wall; polyadenylic-polyuridylic acid; poly (IC); lipopolysaccharide; monophosphoryl lipid A; flagellin; Gardiquimod; Imiquimod; R848; oligonucleosides containing CpG motifs; a CD40 agonist, and 23S ribosomal RNA.

36. The method of claim 35, wherein the agent the augments the immune response is poly (IC).

37. The method of claim 30, further comprising administering at least one immunogenic MHC-I-restricted antigen from a human CMV protein.

38. The method of claim 37, wherein the CMV protein is selected from the group consisting of pp50, pp65, pp150, gB, IE-1, IE-2, US2, US6, UL16, and UL18.

39. The method of claim 38, wherein the at least one MHC-I-restricted antigen comprises the sequence set forth in one of SEQ ID NOs: 34-67.

40. The method of claim 30, wherein prior to introducing the antigen into the tumor, the subject is confirmed as having a preexisting immune response to the antigen.

41. The method of claim 40, wherein confirming the presence of the preexisting immune response comprises identifying a T-cell response to the antigen, in a sample from the subject.

42. The method of claim 30, wherein the administering is via injection.

43. The method of claim 42, wherein the injection is direct into the solid tumor.


SEQUENCE LISTING (67 sequences)

SEQ ID NO: 1 - protein, 15 aa, Human cytomegalovirus
LLQTGIHVRVSQPSL

SEQ ID NO: 2 - protein, 15 aa, Human cytomegalovirus
PLKMLNIPSINVHHY

SEQ ID NO: 3 - protein, 15 aa, Human cytomegalovirus
TRQQNQWKEPDVYYT

SEQ ID NO: 4 - protein, 15 aa, Human cytomegalovirus
EPDVYYTSAFVFPTK

SEQ ID NO: 5 - protein, 15 aa, Human cytomegalovirus
KVYLESFCEDVPSGK

SEQ ID NO: 6 - protein, 15 aa, Human cytomegalovirus
TLGSDVEEDLTMTRN

SEQ ID NO: 7 - protein, 15 aa, Human cytomegalovirus
QPFMRPHERNGFTVL

SEQ ID NO: 8 - protein, 15 aa, Human cytomegalovirus
IIKPGKISHIMLDVA

SEQ ID NO: 9 - protein, 15 aa, Human cytomegalovirus
EHPTFTSQYRIQGKL

SEQ ID NO: 10 - protein, 15 aa, Human cytomegalovirus
YRIQGKLEYRHTWDR

SEQ ID NO: 11 - protein, 15 aa, Human cytomegalovirus
TERKTPRVTGGGAMA

SEQ ID NO: 12 - protein, 15 aa, Human cytomegalovirus
ASTSAGRKRKSASSA

SEQ ID NO: 13 - protein, 15 aa, Human cytomegalovirus
ACTSGVMTRGRLKAE

SEQ ID NO: 14 - protein, 15 aa, Human cytomegalovirus
AGILARNLVPMVATV

SEQ ID NO: 15 - protein, 15 aa, Human cytomegalovirus
KYQEFFWDANDIYRI

SEQ ID NO: 16 - protein, 15 aa, Human cytomegalovirus
PDDYSNTHSTRYVTV

SEQ ID NO: 17 - protein, 15 aa, Human cytomegalovirus
HSRSGSVSQRVTSSQ

SEQ ID NO: 18 - protein, 15 aa, Human cytomegalovirus
FETTGGLVVFWQGIK

SEQ ID NO: 19 - protein, 13 aa, Human cytomegalovirus
YEYVDYLFKRMID

SEQ ID NO: 20 - protein, 20 aa, Human cytomegalovirus
RSYAYIYTTYLLGSNTEYVA

SEQ ID NO: 21 - protein, 20 aa, Human cytomegalovirus
NASYFGENADKFFIFPNYTI

SEQ ID NO: 22 - protein, 20 aa, Human cytomegalovirus
LTFWEASERTIRSEAEDSYH

SEQ ID NO: 23 - protein, 20 aa, Human cytomegalovirus
IRSEAEDSYHFSSAKMTATF

SEQ ID NO: 24 - protein, 20 aa, Human cytomegalovirus
NEQAYQMLLALARLDAEQRA

SEQ ID NO: 25 - protein, 19 aa, Human cytomegalovirus
YRNIEFFTKNSAFPKTTNG

SEQ ID NO: 26 - protein, 19 aa, Human cytomegalovirus
FPKTTNGCSQAMAALQNLP

SEQ ID NO: 27 - protein, 19 aa, Human cytomegalovirus
ARAKKDELRRKMMYMCYRN

SEQ ID NO: 28 - protein, 19 aa, Human cytomegalovirus
SVMKRRIEEICMKVFAQYI

SEQ ID NO: 29 - protein, 19 aa, Human cytomegalovirus
LVKQIKVRVDMVRHRIKEH

SEQ ID NO: 30 - protein, 15 aa, Human cytomegalovirus
VKSEPVSEIEEVAPE

SEQ ID NO: 31 - protein, 19 aa, Human cytomegalovirus
RRKMMYMCYRNIEFFTKNS

SEQ ID NO: 32 - protein, 20 aa, Human cytomegalovirus
QLNRHSYLKDSDFLDAALDF

SEQ ID NO: 33 - protein, 20 aa, Human cytomegalovirus
QGDKYESWLRPLVNVTRRDG

SEQ ID NO: 34 - protein, 9 aa, Human cytomegalovirus
NLVPMVATV

SEQ ID NO: 35 - protein, 8 aa, Human cytomegalovirus
FPTKDVAL

SEQ ID NO: 36 - protein, 9 aa, Human cytomegalovirus
VTEHDTLLY

SEQ ID NO: 37 - protein, 9 aa, Human cytomegalovirus
ELKRKMMYM

SEQ ID NO: 38 - protein, 9 aa, Human cytomegalovirus
VLEETSVML

SEQ ID NO: 39 - protein, 10 aa, Human cytomegalovirus
AYAQKIFKIL

SEQ ID NO: 40 - protein, 9 aa, Human cytomegalovirus
IMREFNSYK

SEQ ID NO: 41 - protein, 9 aa, Human cytomegalovirus
QYDPVAALF

SEQ ID NO: 42 - protein, 9 aa, Human cytomegalovirus
DIYRIFAEL

SEQ ID NO: 43 - protein, 10 aa, Human cytomegalovirus
TPRVTGGGAM

SEQ ID NO: 44 - protein, 9 aa, Human cytomegalovirus
QIKVRVDMV

SEQ ID NO: 45 - protein, 11 aa, Human cytomegalovirus
YSEHPTFTSQY

SEQ ID NO: 46 - protein, 9 aa, Human cytomegalovirus
FEQPTETPP

SEQ ID NO: 47 - protein, 9 aa, Human cytomegalovirus
ARVYEIKCR

SEQ ID NO: 48 - protein, 9 aa, Human cytomegalovirus
QMWQARLTV

SEQ ID NO: 49 - protein, 12 aa, Human cytomegalovirus
PFTSQYRIQGKL

SEQ ID NO: 50 - protein, 12 aa, Human cytomegalovirus
CPSQEPMSIYVY

SEQ ID NO: 51 - protein, 9 aa, Human cytomegalovirus
TRATKMQVI

SEQ ID NO: 52 - protein, 10 aa, Human cytomegalovirus
ERAWALKNPH

SEQ ID NO: 53 - protein, 9 aa, Human cytomegalovirus
GPISGHVLK

SEQ ID NO: 54 - protein, 8 aa, Human cytomegalovirus
DALPGPCI

SEQ ID NO: 55 - protein, 9 aa, Human cytomegalovirus
KMQVIGDQY

SEQ ID NO: 56 - protein, 9 aa, Human cytomegalovirus
CEDVPSGKL

SEQ ID NO: 57 - protein, 9 aa, Human cytomegalovirus
LYLCCGITL

SEQ ID NO: 58 - protein, 9 aa, Human cytomegalovirus
VYVTVDCNL

SEQ ID NO: 59 - protein, 10 aa, Human cytomegalovirus
LYTSRMVTNL

SEQ ID NO: 60 - protein, 9 aa, Human cytomegalovirus
IPSINVHHY

SEQ ID NO: 61 - protein, 9 aa, Human cytomegalovirus
QAIRETVEL

SEQ ID NO: 62 - protein, 9 aa, Human cytomegalovirus
PGKISHIML

SEQ ID NO: 63 - protein, 9 aa, Human cytomegalovirus
YEQHKITSY

SEQ ID NO: 64 - protein, 10 aa, Human cytomegalovirus
TENGSFVAGY

SEQ ID NO: 65 - protein, 10 aa, Human cytomegalovirus
QEFFWDANDI

SEQ ID NO: 66 - protein, 8 aa, Human cytomegalovirus
YRNMIIHA

SEQ ID NO: 67 - protein, 9 aa, Human cytomegalovirus
YAYIYTTYL